(19) United States
(12) Patent Application Publication
(10) Pub. No.: US 2017/0334837 A1
(43) Pub. Date: Nov. 23, 2017

(54) DIAMINE COMPOUND, AND HEAT-RESISTANT RESIN OR HEAT-RESISTANT RESIN PRECURSOR USING SAME

(71) Applicant: TORAY INDUSTRIES, INC., Tokyo (JP)

(72) Inventors: Yusuke KOMORI, Otsu-shi (JP); Satoshi KAMEMOTO, Otsu-shi (JP); Kazuto MIYOSHI, Otsu-shi (JP)

(73) Assignee: TORAY INDUSTRIES, INC., Tokyo (JP)

(21) Appl. No.: 15/670,447

(22) Filed: Aug. 7, 2017

Related U.S. Application Data
(63) Continuation of application No. PCT/JP2016/059413, filed on Mar. 24, 2016.

(30) Foreign Application Priority Data
Mar. 27, 2015 (JP) 2015-066007

(51) Int. Cl. C07C 235/64; C08G 73/14; H01L 51/00; G03F 7/40; G03F 7/32; C08G 69/42; G03F 7/037; G03F 7/031; G03F 7/023; G03F 7/004; G03F 7/039; G03F 7/20; H01L 27/32

(52) U.S. Cl. CPC C07C 235/64; G03F 7/039; C08G 73/14; H01L 51/0035; G03F 7/40; G03F 7/322; G03F 7/2004; C08G 69/42; G03F 7/037; G03F 7/031; G03F 7/0233; G03F 7/0048; H01L 27/3258; H01L 2227/323

(57) ABSTRACT

Provided are a photosensitive resin composition which has excellent pattern processabilities (high sensitivity and high resolution) and is excellent in chemical resistance and thermal resistance after thermally treated; a heat-resistant resin or heat-resistant resin precursor used for the composition; and a diamine compound which is a raw material of the resin and the precursor. The diamine compound is a diamine compound represented by a general formula (1).

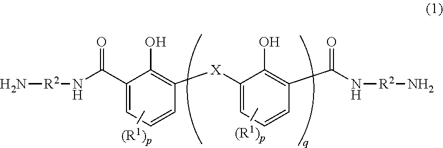

### TECHNICAL FIELD

The present invention relates to a novel diamine compound, a heat-resistant resin or heat-resistant resin precursor including this compound, and a photosensitive resin composition including the heat-resistant resin or heat-resistant resin precursor. More specifically, the invention relates to a photosensitive resin composition suitable for, e.g., a surface protecting film or an interlayer dielectric film of a semiconductor element, or an insulating layer of an organic electroluminescence element.

### BACKGROUND ART

Heat-resistant resins such as polyimide and polybenzoxazole have excellent thermal resistance and electrically insulating property to be used for, e.g., a surface protecting film or an interlayer dielectric film of a semiconductor element such as an LSI, or an insulating layer of an organic electroluminescence element. In recent years, with an advance of microfabrication of semiconductor elements, a resolution of several micrometers and a higher sensitivity are required also for their surface protecting film or interlayer dielectric films. In such a usage, a positive type photosensitive polyimide has been used which can be finely worked and is excellent in sensitivity.

As a means of using any one of these heat-resistant resins to yield a photosensitive resin composition having high pattern processabilities, a positive type photosensitive resin composition has been developed which contains a novolak resin, which is widely used as a resin for photoresists (see, for example, Patent Documents 1 to 4). However, the positive type photosensitive resin composition, which contains the heat-resistant resin and the novolak resin, has a problem of being poor in chemical resistance and thermal resistance after thermally treated.

Another means is also known in which a diamine containing a phenol hydroxyl group is used to introduce a phenolic compound into a polyimide resin skeleton, thereby improving the resultant in pattern processabilities (see, for example, Patent Documents 5 and 6). However, even the resultant, which is a positive type photosensitive resin composition in which the polyimide obtained from the diamine compound is used, has a problem of being insufficient in chemical resistance and thermal resistance after thermally treated.

### PRIOR ART DOCUMENTS

### Patent Documents

Patent Literature 1: JP 2005-62764 A

Patent Literature 2: JP 2005-250160 A

Patent Literature 3: JP 2005-352004 A

Patent Literature 4: JP 2006-285037 A

Patent Literature 5: JP 2003-327646 A

Patent Literature 6: JP 2004-317725 A

### SUMMARY OF THE INVENTION

### Problems to be Solved by the Invention

An object of the present invention is to provide a photosensitive resin composition which has excellent pattern processabilities (high sensitivity and high resolution) and is excellent in chemical resistance and thermal resistance after thermally treated; a heat-resistant resin or heat-resistant resin precursor used for the composition; and a diamine compound which is a raw material of the resin and the precursor.

### Solutions to the Problems

In order to solve the above-mentioned problems, the photosensitive resin composition of the present invention, a heat-resistant resin or heat-resistant resin precursor used for the composition, and a diamine compound which is a raw material of the resin and the precursor are as follows:
[1] A diamine compound represented by the general formula (1):

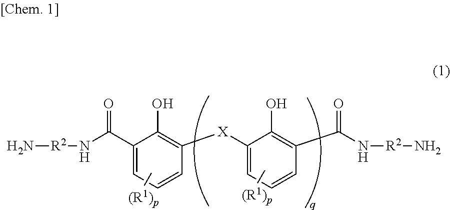

In the general formula (1), each R¹ represents an alkyl group having 1 to 5 carbon atoms; each p represents an integer of 0 to 2; q represents an integer of 0 to 100; each R² represents any case of a bivalent aliphatic group, alicyclic group or aromatic group, any case of a bivalent organic group in which plural aromatic groups are bonded to each other through a single bond, or any case of a bivalent organic group in which plural aromatic groups are bonded to each other through —O—, —CO—, —SO₂—, —CH₂—, —C(CH₃)₂—, or —C(CF₃)₂ wherein each F is fluorine; X represents —O—, —S—, —CO—, —SO₂—, —CH₂, —C (CH₃)₂—, —C(CH₃) (C₂H₅)—, or —C(CF₃)₂— wherein each F is fluorine.
[2] A heat-resistant resin or heat-resistant resin precursor, having a structure originating from the diamine compound recited in item [1].
[3] The heat-resistant resin or heat-resistant resin precursor according to item [2], including at least one selected from polyimides, polybenzoxazoles, polybenzoimidazoles, and polybenzothiazoles; and respective precursors of these polymers, and copolymers of these polymers.
[4] The heat-resistant resin or heat-resistant resin precursor according to item [2] or [3], having at least one selected from respective structures represented by the general formulae (2), (3), and (5):

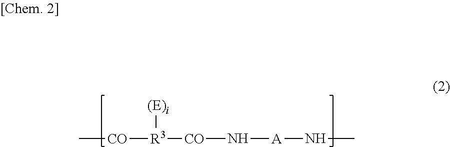

In the general formula (2), R³ represents a bivalent to hexavalent organic group having 2 to 30 carbon atoms; E represents any one of OR⁴, SO₃R⁴, CONR⁴R⁵, COOR⁴, and SO₂NR⁴R⁵ wherein R⁴ and R⁵ each independently represent a hydrogen atom or a monovalent hydrocarbon group having 1 to 20 carbon atoms; i represents an integer of 0 to 4; and A represents a structure represented by a general formula (4).

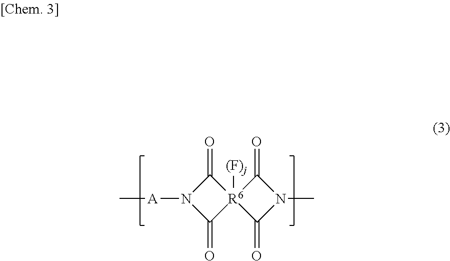

In the general formula (3), R⁶ represents a tetravalent to octavalent organic group having 2 to 30 carbon atoms; F represents any one of OR⁷, SO₃R⁷, CONR⁷R⁸, COOR⁷, and SO₂NR⁷R⁸ wherein R⁷ and R⁸ each independently represent a hydrogen atom or a monovalent hydrocarbon group having 1 to 10 carbon atoms; j represents an integer of 0 to 4; and A represents a structure represented by the general formula (4):

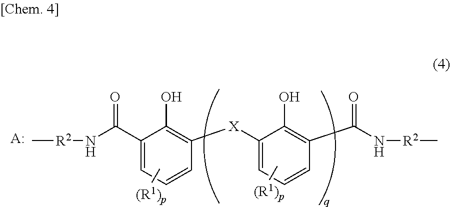

In the general formula (4), each R¹ represents an alkyl group having 1 to 5 carbon atoms; each p is an integer of 0 to 2; q represents an integer of 0 to 100; each R² represents any case of a bivalent aliphatic group, alicyclic group, or aromatic group, any case of a bivalent organic group in which plural aromatic group are bonded to each other through a single bond, or any case of a bivalent organic group in which plural aromatic groups are bonded to each other through —O—, —CO—, —SO₂—, —CH₂—, —C(CH₃)₂—, or —C(CF₃)₂— wherein each F is fluorine; and X represents —O—, —S—, —CO—, —SO₂—, —CH₂—, —C(CH₃)₂—, —C(CH₃) (C₂H₅)—, or —C(CF₃)₂— wherein each F is fluorine; and

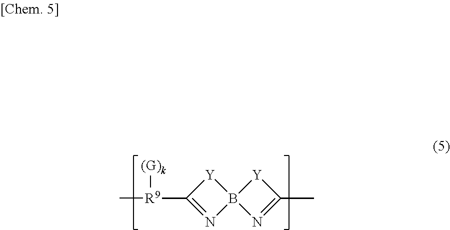

In the general formula (5), R⁹ represents a bivalent to hexavalent organic group having 2 to 30 carbon atoms; G represents any one of OR¹⁰, SO₃R¹⁰, CONR¹⁰R¹¹, COOR¹⁰, and SO₂NR¹⁰R¹¹ wherein R¹⁰ and R¹¹ each represent a hydrogen atom or a monovalent hydrocarbon group having 1 to 10 carbon atoms; k represent an integer of 0 to 4; B represents a structure represented by a general formula (6), and each Y represents NH, O or S;

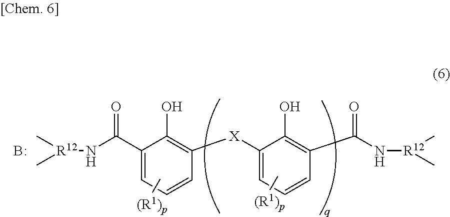

In the general formula (6), each R¹ represents an alkyl group having 1 to 5 carbon atoms; each p represents an integer of 0 to 2; q represents an integer of 0 to 100; each R¹² represents any case of a trivalent aliphatic group, alicyclic group, or aromatic group, any case of a trivalent organic group in which plural aromatic groups are bonded to each other through a single bond, or any case of a trivalent organic group in which plural aromatic groups are bonded to each other through —O—, —CO—, —SO₂—, —CH₂—, —C(CH₃)₂—, or —C(CF₃)₂ wherein each F is fluorine; and X represents —O—, —S—, —CO—, —SO₂—, —CH₂—, —C(CH₃)₂—, —C(CH₃) (C₂H₅)—, or —C(CF₃)₂—.
[5] A photosensitive resin composition, including the heat-resistant resin or heat-resistant resin precursor (a) recited in any one of items [2] to [4], and further including a photosensitive compound (b) and a solvent (c).
[6] The photosensitive resin composition according to item [5], wherein the photosensitive compound (b) is a quinonediazide compound (b1).
[⁷] The photosensitive resin composition according to item [5], wherein the photosensitive compound (b) is a photopolymerization initiator (b2).
[8] The photosensitive resin composition according to item [7], further including a radical polymerizable compound (d).
[9] The photosensitive resin composition according to any one of items [5] to [8], further including an alkoxymethyl-group-containing compound, and/or a cyclic-polyether-structure-having compound (e).
[10] A cured film, wherein the photosensitive resin composition recited in any one of items [5] to [9] is cured.
[11] An element, including the cured film recited in item [10].
[12] An organic EL display device, wherein the cured film recited in item [10] is located over at least one of a planarizing layer over a driving circuit, and an insulating layer over a first electrode.
[13] A method for producing an organic EL display device, using the photosensitive resin composition recited in any one of items [5] to [9], including:

the step of applying the photosensitive resin composition onto a substrate to form a photosensitive resin film; and

the step of subjecting the photosensitive resin film to drying, exposure to light, development, and heating treatment.

### Effects of the Invention

The present invention makes it possible to yield a photosensitive resin composition which has a high sensitivity and a high resolution and is excellent in chemical resistance and thermal resistance after thermally treated; a heat-resistant resin or heat-resistant resin precursor used for the composition; and a diamine compound which is a raw material of the heat-resistant resin or heat-resistant resin precursor.

### EMBODIMENTS OF THE INVENTION

Hereinafter, the present invention will be described in detail.

### <Diamine Compound>

The diamine compound of the present invention is a compound represented by a general formula (1), and is a diamine compound having a phenolic structure.

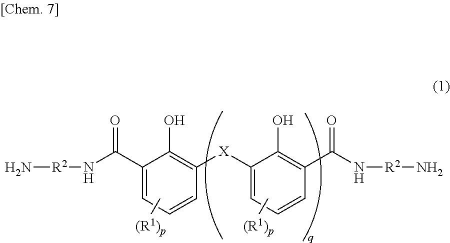

In the general formula (1), each R¹ represents an alkyl group having 1 to 5 carbon atoms; each p represents an integer of 0 to 2; q represents an integer of 0 to 100; each R² represents any case of a bivalent aliphatic group, alicyclic group or aromatic group, any case of a bivalent organic group in which plural aromatic groups are bonded to each other through a single bond, or any case of a bivalent organic group in which plural aromatic groups are bonded to each other through —O—, —CO—, —SO₂—, —CH₂—, —C(CH₃)₂—, or —C(CF₃)₂— wherein each F is fluorine; X represents —O—, —S—, —CO—, —SO₂—, —CH₂—, —C(CH₃)₂—, —C(CH₃) (C₂H₅) or —C(CF₃)₂— wherein each F is fluorine.

In the general formula (1), each R¹ represents an alkyl group having 1 to 5 carbon atoms. From the viewpoint of an improvement in water-resistant property, each R¹ is preferably a methyl, ethyl, n-propyl or t-butyl group, more preferably a methyl group. Each p represents an integer of 0 to 2. In order to improve in solubility in solvents, each p is preferably an integer of 1 or 2, more preferably 1. The symbol q represents an integer of 0 to 100. In order to improve in solubility in solvents, q is preferably from 1 to 10, more preferably from 1 to 5. Each R² represents a bivalent to hexavalent organic group having 2 to 30 carbon atoms, and each R² is preferably an organic group having an aromatic ring and/or an aliphatic ring to improve in thermal resistance.

Examples of a diamine component constituting NH₂—R²—NH—in the general formula (1) include hydroxyl-group-containing diamines such as bis(3-amino-4-hydroxyphenyl)hexafluoropropane (BAHF), bis(3-amino-4-hydroxyphenyl)sulfone, bis(3-amino-4-hydroxyphenyl)propane, bis(3-amino-4-hydroxyphenyl)methylene, bis(3-amino-4-hydroxyphenyl) ether, bis(3-amino-4-hydroxy)biphenyl, and bis(3-amino-4-hydroxyphenyl)fluorene; carboxyl-group-containing diamines such as 3, 5-diaminobenzoic acid, and 3-carboxyl-4,4′-diaminodiphenyl ether; sulfonic-acid-containing diamines such as 3-sulfonic acid-4,4′-diaminodiphenyl ether; and dithiohydroxyphenyldiamine. Other examples thereof include 3,4′-diaminodiphenyl ether, 4,4′-diaminodiphenyl ether, 3,4′-diaminodiphenylmethane, 4,4′-diaminodiphenylmethane, 3,4′-diaminodiphenylsulfone, 4,4′-diaminodiphenylsulfone, 3,4′-diaminodiphenyl sulfide, 4,4′-diaminodiphenyl sulfide, 1,4-bis(4-aminophenoxy)benzene, benzine, m-phenylenediamine, p-phenylenediamine, 1,5-naphthalenediamine, 2,6-naphthalenediamine, bis(4-aminophenoxyphenyl)sulfone, bis(3-aminophenoxyphenyl)sulfone, bis(4-aminophenoxy)biphenyl, bis{4-(4-aminophenoxy)phenyl} ether, 1,4-bis(4-aminophenoxy)benzene, 2,2′-dimethyl-4,4′-diaminobiphenyl, 2,2′-diethyl-4,4′-diaminobiphenyl, 3,3′-dimethyl-4,4′-diaminobiphenyl, 3,3′-diethyl-4,4′-diaminobiphenyl, 2,2′,3,3′-tetramethyl-4,4′-diaminobiphenyl, 3,3′,4,4′-tetramethyl-4,4′-diaminobiphenyl, and 2,2′-di(trifluoromethyl)-4,4′-diaminobiphenyl; and compounds each obtained by substituting an aromatic ring of any one of these examples with an alkyl group or a halogen atom. Additional examples thereof include aliphatic cyclohexyldiamine, methylenebiscyclohexylamine, hexamethylenediamine, diaminocyclohexane, diaminoadamantane, bis(aminocyclohexyl)propane, and cyclohexanebis(methylamine).

These diamines may be substituted with a group, for example, a monovalent group having 1 to 10 carbon atoms such as a methyl or ethyl group, or a fluoroalkyl group having 1 to 10 carbon atoms such as a trifluoromethyl group, or with a radical of F, Cl, Br or I. Out of these examples, aromatic diamines are preferred since the diamines improve the resultant polymer in thermal resistance. Any bisaminophenol compound of the present invention that is obtained from a diamine compound having a hydroxyl group and an amino group which are adjacent to each other has a hydroxyl group and an amide group which are adjacent to each other. About a heat-resistant resin or heat-resistant resin precursor obtained from the bisaminophenol compound, the amide group and the hydroxyl group undergo dehydration reaction while the resin or precursor is thermally treated, so that these groups turn to an oxazole ring. Thus, the resultant polymer is improved in thermal resistance and solvent resistance. Moreover, through the hydroxyl group, the resultant photosensitive resin composition is improved in sensitivity and resolution. Thus, more preferred is the diamine compound having a hydroxyl group and an amino group which are adjacent to each other.

X represents —O—, —S—, —CO—, —SO₂—, —CH₂—, —C(CH₃)₂—, —C(CH₃) (C₂H₅)—, or —C(CF₃)₂—. In a case where X is —CH₂—, a known reaction using a phenolic compound and formalin can be used to conduct the step concerned when the diamine compound is produced. Thus, this case is preferred since the production process of the diamine compound is easy.

### <Method for Producing Diamine Compound Represented by General Formula (1)>

The diamine compound represented by the general formula (1) (hereinafter referred to also as the bisaminophenol compound) can be produced in accordance with a known method for producing a diamine compound. The production method of the bisaminophenol compound is not particularly limited. When X in the general formula (1) is CH₂, the bisaminophenol compound can be produced by, for example, a method described below.

In a first step, formalin is caused to react with a phenolic compound represented by a general formula (7) illustrated below in the presence of a base to produce a dimethylolphenol derivative in which two —CH₂OH groups are bonded to each other, as shown by a general formula (8) illustrated below. The number q can be varied in accordance with, for example, properties of the phenolic compound of the starting compound shown in the general formula (7) and conditions for the reaction. The number q is preferably from 0 to 10, more preferably from 0 to 5 since the diamine is improved in solubility in solvents.

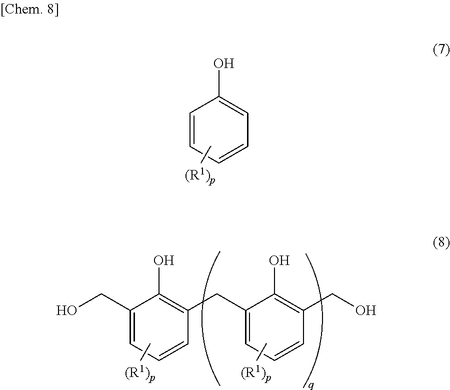

In the general formulae (7) and (8), each R¹ represents an alkyl group having 1 to 5 carbon atoms; each p is an integer of 0 to 2; and q is an integer of 0 to 100.

Next, through a second step in which an oxidizer is caused to act on the two —CH₂OH groups of this dimethylolphenol derivative, the corresponding phenol dicarboxylic acid derivative is yielded. In a third step, a chlorinating agent is used to convert the phenol dicarboxylic acid derivative to the corresponding phenol dicarboxylic acid chloride derivative. Thereafter, this derivative is coupled with a diamine compound in a fourth step to succeed in yielding a bisaminophenol compound of the present invention. This fourth step makes use of a method of performing the de-hydrochlorination concerned in the presence of a de-hydrochlorinating agent, a method of using a tertiary amine such as triethylamine in an aprotic polar solvent to attain the de-hydrochlorination, or a method of using an ion exchange resin in an aprotic polar solvent to attain the de-hydrochlorination. From the viewpoint of the purity and the yield of the target substance, preferred is the method of causing the compounds concerned to react with each other in the presence of a de-hydrochlorinating agent. The following will demonstrate reactions in these steps, giving typical compounds as examples.

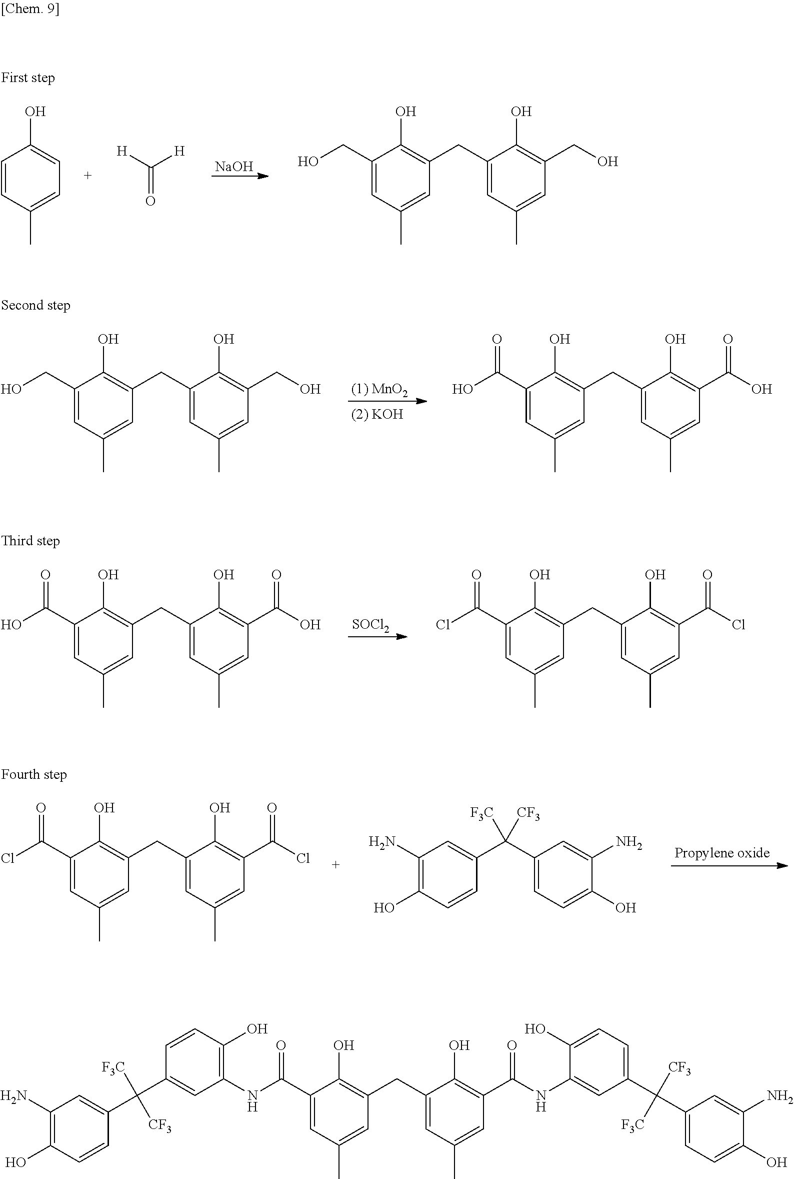

Specific reaction conditions for the first step are as follows: Into a reactor equipped with a stirrer, a thermostat, a condenser and a dropping funnel are charged a phenolic compound as a raw material, and an aqueous formalin solution containing formalin in an amount 2 to 4 times the mole of this phenolic compound (formalin concentration: preferably about 50% by mass). While the solution is stirred, an alkali is dropwise added thereto at 0 to 50° C. for 1 to 2 hours. The alkali is preferably, for example, an aqueous solution of an alkali such as sodium hydroxide or potassium hydroxide. For example, an aqueous sodium hydroxide solution can be used which has a concentration of, e.g., about 30% by mass. The alkali is used in such a proportion that the mole thereof is substantially equal to that of the phenolic compound.

Thereafter, the temperature of the reaction system is raised, and the reactive components are caused to react with each other, for example, at a reaction temperature of 20 to 80° C. for a reaction period of 2 to 4 hours. Next, the reaction system is cooled preferably to 30° C. or lower, and then neutralized with an acid to precipitate a product. The acid is not particularly limited, and may be, for example, an aqueous acetic acid solution having a concentration of about 10% by mass. Next, the system is filtrated, and then the product is washed with water. The system is dried under a reduced pressure preferably at a temperature of 50° C. or lower to yield a product (dimethylolphenol derivative).

Subsequently, in the second step, in a reactor equipped with a stirrer, a thermostat, a condenser and a dropping funnel, the dimethylolphenol derivative yielded in the first step is dissolved in a reaction solvent. Thereinto is charged a first oxidizer in an amount 2 to 50 times the mole number of the dimethylolphenol derivative. The system is then heated and stirred preferably at 40 to 100° C., more preferably at 60 to 100° C. preferably for 2 to 24 hours, more preferably for 10 to 24 hours. By adjusting the temperature and the period into such ranges, side reactions can be prevented to cause the raw materials wholly to react with each other. The resultant filtrate is concentrated and dried to yield a product. Examples of the reaction solvent include halogens such as chloroform, dichloroethane, dichloromethane, trichloroethylene, and tetrachloroethylene; ketones such as methyl isobutyl ketone, diisobutyl ketone, cyclohexanone, methyl ethyl ketone, and acetone; esters such as ethyl acetate, butyl acetate, and isobutyl acetate; and ethers such as tetrahydrofuran, dimethoxyethane, diethoxyethane, dibutyl ether, and diethylene glycol dimethyl ether. Out of these examples, chloroform is preferred from the viewpoint of the dissolving performance thereof. The use amount of the reaction solvent is preferably in a range of 100 to 5000 parts by mass for 100 parts by mass of the dimethylolphenol derivative from the viewpoint of the dissolving performance.

Examples of the first oxidizer include manganese dioxide, potassium permanganate, and sodium hypochlorite. These oxidizers may be used singly or in combination of two or more thereof. Out of the examples, manganese dioxide is more preferred since the system is easily post-treated after the reaction. Furthermore, the resultant product is heated and stirred preferably at 180 to 250° C., more preferably at 200 to 250° C. in the presence of a second oxidizer in an amount 2 to 50 times the mole number of the dimethylolphenol derivative preferably for 1 to 4 hours, more preferably for 1 to 2 hours. By setting the temperature and the period into such ranges, side reactions can be prevented to cause the raw materials wholly to react with each other. The reaction substances are dissolved into pure water, and an acid is added thereto to set the pH of the system to 1. Thereafter, the resultant precipitation is filtrated, washed with pure water, and then dried under a reduced pressure to yield a product (phenol dicarboxylic acid derivative). Examples of the second oxidizer include sodium hydroxide, potassium hydroxide, and calcium hydroxide. These may be used singly or in combination of two or more thereof. Out of these examples, potassium hydroxide is more preferred. The acid is not particularly limited, and may be, for example, hydrochloric acid.

In the third step, into a reactor are charged the phenol dicarboxylic acid derivative yielded in the second step, and a chlorinating agent in an amount 2 to 10 times the mole number of this phenol dicarboxylic acid derivative. These components are caused to react with each other preferably at −10 to 40° C., more preferably 10 to 30° C. preferably for 2 to 24 hours, more preferably 2 to 4 hours. By setting the temperature and the period into such ranges, side reactions can be prevented to cause the raw materials wholly to react with each other. The reaction solution is filtrated into a filtrate and a solid, and the filtrate is concentrated to yield a product (phenol dicarboxylic acid chloride derivative). Examples of the chlorinating agent include thionyl chloride, oxalyl chloride, phosphoryl chloride, phosphorus trichloride, phosphorus pentachloride, and N-chlorosuccinimide. Thionyl chloride is more preferred since the system is easily post-treated after the reaction.

This reaction can be conducted in the presence or absence of a solvent. Examples of the reaction solvent include aromatic hydrocarbons such as benzene, toluene and xylene; halogens such as methylene chloride, chloroform, and 1,2-dichloroethane; and ethers such as diethyl ether, diisopropyl ether, tetrahydrofuran, and dioxane. The reaction is conducted preferably in the absence of any solvent since the system is easily post-treated.

In the fourth step, the diamine compound is dissolved into a reaction solvent and a de-hydrochlorinating agent. Thereto is dropwise added a solution in which the phenol dicarboxylic acid chloride derivative yielded in the third step is dissolved in a reaction solvent. After the end of the addition, the reactive components are caused to react with each other preferably at −15 to 40° C., more preferably at −10 to 30° C. preferably for 1 to 10 hours, more preferably for 3 to 5 hours. Thereafter, the temperature of the system is returned to room temperature. By setting the temperature and the period into such ranges, side reactions can be prevented to cause the raw materials wholly to react with each other. The resultant precipitated white solid is isolated by filtration, and dried. In this way, a bisaminophenol compound of the present invention can be yielded. The de-hydrochlorinating agent is preferably a compound having an epoxy group since the system is easily post-treated after the reaction. Examples thereof include propylene oxide, and glycidyl methyl ether. The use amount of the de-hydrochlorinating agent is preferably a mole equivalent to or more than each mole of the aromatic halide nitro compound, and is more preferably an amount 5 to 10 times (both inclusive) the mole number of the nitro compound since a by-product does not easily generate. [0039]

Examples of the reaction solvent include ketones such as methyl isobutyl ketone, diisobutyl ketone, cyclohexanone, methyl ethyl ketone, and acetone; esters such as ethyl acetate, butyl acetate, and isobutyl acetate; and ethers such as tetrahydrofuran, dimethoxyethane, diethoxyethane, dibutyl ether, and diethylene glycol dimethyl ether. Out of these examples, acetone is preferred from the viewpoint of the dissolving performance and versatility thereof. These may be used singly or in the form of a mixture of two or more thereof. The use amount of the reaction solvent is preferably in a range of 100 to 5000 parts by mass for 100 parts by mass of the fluorenylidene bisaminophenol compound from the viewpoint of the dissolving performance.

Examples of the diamine compound include hydroxyl-group-containing diamines such as bis(3-amino-4-hydroxyphenyl)hexafluoropropane (BAHF), bis(3-amino-4-hydroxyphenyl)sulfone, bis(3-amino-4-hydroxyphenyl)propane, bis(3-amino-4-hydroxyphenyl)methylene, bis(3-amino-4-hydroxyphenyl) ether, bis(3-amino-4-hydroxy)biphenyl, and bis(3-amino-4-hydroxyphenyl)fluorene; carboxyl-group-containing diamines such as 3, 5-diaminobenzoic acid, and 3-carboxyl-4,4′-diaminodiphenyl ether; sulfonic-acid-containing diamines such as 3-sulfonic acid-4,4′-diaminodiphenyl ether; and dithiohydroxyphenyldiamine.

Other examples thereof include 3,4′-diaminodiphenyl ether, 4,4′-diaminodiphenyl ether, 3,4′-diaminodiphenylmethane, 4,4′-diaminodiphenylmethane, 3,4′-diaminodiphenylsulfone, 4,4′-diaminodiphenylsulfone, 3,4′-diaminodiphenyl sulfide, 4,4′-diaminodiphenyl sulfide, 1,4-bis(4-aminophenoxy)benzene, benzine, m-phenylenediamine, p-phenylenediamine, 1,5-naphthalenediamine, 2,6-naphthalenediamine, bis(4-aminophenoxyphenyl)sulfone, bis(3-aminophenoxyphenyl)sulfone, bis(4-aminophenoxy)biphenyl, bis{4-(4-aminophenoxy)phenyl} ether, 1,4-bis(4-aminophenoxy)benzene, 2,2′-dimethyl-4,4′-diaminobiphenyl, 2,2′-diethyl-4,4′-diaminobiphenyl, 3,3′-dimethyl-4,4′-diaminobiphenyl, 3,3′-diethyl-4,4′-diaminobiphenyl, 2,2′,3,3′-tetramethyl-4,4′-diaminobiphenyl, 3,3′,4,4′-tetramethyl-4,4′-diaminobiphenyl, and 2,2′-di(trifluoromethyl)-4,4′-diaminobiphenyl; and compounds each obtained by substituting an aromatic ring of any one of these examples with an alkyl group or a halogen atom.

Additional examples thereof include aliphatic cyclohexyldiamine, methylenebiscyclohexylamine, hexamethylenediamine, diaminocyclohexane, diaminoadamantane, bis(aminocyclohexyl)propane, and cyclohexanebis(methylamine).

These diamines may be substituted with a group, for example, a monovalent group having 1 to 10 carbon atoms, such as a methyl or ethyl group, or a fluoroalkyl group having 1 to 10 carbon atoms, such as a trifluoromethyl group, or with a radical of F, Cl, Br or I. Out of the above-mentioned examples, aromatic diamines are preferred since the diamines improve the resultant polymer in thermal resistance. Any bisaminophenol compound of the present invention that is obtained from a diamine compound having a hydroxyl group and an amino group which are adjacent to each other has a hydroxyl group and an amide group which are adjacent to each other. About a heat-resistant resin or heat-resistant resin precursor obtained from the bisaminophenol compound, the amide group and the hydroxyl group undergo dehydration reaction while the resin or precursor is thermally treated, so that these groups turn to an oxazole ring. Thus, the resultant polymer is improved in thermal resistance and solvent resistance. Moreover, through the hydroxyl group, the resultant photosensitive resin composition is improved in sensitivity and resolution. Thus, more preferred is the diamine compound having a hydroxyl group and an amino group which are adjacent to each other.

### <Heat-Resistant Resin Precursor or Heat-Resistant Resin (a) Used in the Invention>

The heat-resistant resin or heat-resistant resin precursor (a) used in the present invention preferably has as a main component thereof, at least one selected from respective structures represented by the general formulae (2), (3), and (5). The main component denotes that in the heat-resistant resin or heat-resistant resin precursor (a), the proportion of at least one selected from respective structures represented by the general formulae (2), (3), and (5) is 50% or more by mole, preferably 70% or more by mole, more preferably 90% or more by mole. The matter that the component (a) has, as the main component, the above-mentioned structure makes it possible to yield a cured film high in thermal resistance when the photosensitive resin composition of the present invention has been cured. A resin having the structural unit represented by the general formula (2) is a resin having, at a main chain thereof, an amide bond. Examples thereof include any polyamic acid, which is a polyimide precursor, polyamic acid ester, polyhydroxyamide, which can be a polybenzoxazole precursor, polyaminoamide, polyamide, and polyamideimide. It is sufficient for the resin to be a resin having the structural unit of the general formula (2) besides any one of these examples. Out of these examples, preferred are, for example, any polyamic acid, polyamic acid ester, and polyhydroxyamide. More preferred are any polyamic acid and polyamic acid ester. When these resins are each fired even at a low temperature of 250° C. or lower, the resin sufficiently forms imide rings to be further improved in chemical resistance in the low-temperature firing.

Instead of the structural unit represented by the general formula (2), or in combination therewith, a resin having the imide ring structure represented by the general formula (3) may be used. The respective structural units represented by the general formulae (2) and (3) may be used singly, or in the form of a mixture or copolymer of the two or more thereof.

Instead of the structural unit represented by the general formula (2) or (3), or in combination therewith, a resin having the structural unit represented by the general formula (5) may be used. The resin having the structural unit represented by the general formula (5) may be a resin having, in a main chain structure thereof, a ring structure such as an oxazole ring, an imidazole ring or a thiazole ring. Specific examples thereof include any polybenzoxazole, polybenzimidazole, and polybenzthiazole. The respective structural units represented by the general formulae (2), (3) and (5) may be used singly, or in the form of a mixture or copolymer of two or more thereof.

A preferred component (a) in the present invention is a resin having the structural unit represented by the general formula (2), a resin having the respective structural units represented by the general formulae (2) and (3), or a resin having the respective structural units represented by the general formulae (2) and (5) since the photosensitive resin composition is improved in sensitivity. A more preferred component (a) therein is a resin having the structural unit represented by the general formula (2).

A polyimide used preferably in the present invention can be yielded by causing, e.g., a tetracarboxylic acid, the corresponding tetracarboxylic dianhydride, or a tetracarboxylic acid diester dichloride as an acid component to react with a diamine, the corresponding diisocyanate compound, or a trimethylsilylated diamine. Any polyimide can be generally yielded by subjecting a polyamic acid, which is a polyimide precursor yielded by causing a tetracarboxylic dianhydride to react with a diamine, to heating or chemical treatment with, e.g., an acid or base, thereby dehydrating and ring-closing the polyamic acid. In the present invention, a polyamic acid or a polyimide can be used, and further, for example, a polyamic acid ester, a polyamic acid amide, or a polyisoimide, which is a different polyimide precursor, can also be used.

A polybenzoxazole used preferably in the present invention can be yielded by causing a bisaminophenol to react with, e.g., a dicarboxylic acid, the corresponding dicarboxylic acid chloride, or an active dicarboxylic acid ester as an acid component. In general, any polybenzoxazole can be yielded by subjecting a polyhydroxyamide, which is a polybenzoxazole precursor yielded by causing a bisaminophenol compound to react with a dicarboxylic acid, to heating or chemical treatment with, e.g., phosphoric anhydride, abase or a carbodiimide compound, thereby dehydrating and ring-closing the polyhydroxyamide.

The polybenzothiazole can be yielded by causing bisaminothiophenol to react with, e.g., a dicarboxylic acid, the corresponding dicarboxylic acid chloride, or an active dicarboxylic acid ester as an acid component. In general, any polybenzothiazole can be yielded by subjecting a polythiohydroxyamide, which is a polybenzthiazole precursor yielded by causing a bisaminothiophenol compound to react with a dicarboxylic acid, to heating or chemical treatment with, e.g., phosphoric anhydride, a base or a carbodiimide compound, thereby dehydrating and ring-closing the polythiohydroxyamide.

The polybenzimidazole can be yielded by causing a tetramine to react with, e.g., a dicarboxylic acid, the corresponding dicarboxylic acid chloride, or an active dicarboxylic acid ester as an acid component. In general, any polybenzimidazole can be yielded by subjecting a polyaminoamide, which is a polybenzimidazole precursor yielded by causing a bisaminophenol compound to react with a dicarboxylic acid, to heating or chemical treatment with, e.g., phosphoric anhydride, a base or a carbodiimide compound, thereby dehydrating and ring-closing the polyaminoamide.

### <Heat-Resistant Resin Precursor Including Structural Unit Represented by General Formula (2) as Main Component>

The following will describe a heat-resistant resin precursor including the structural unit represented by the general formula (2) as a main component.

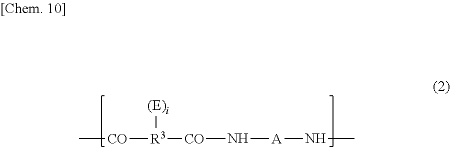

In the general formula (2), R³ represents a bivalent to hexavalent organic group having 2 to 30 carbon atoms, and is preferably a group having an aromatic ring and/or an aliphatic ring.

Examples of a dicarboxylic acid as an acid component constituting —CO—R³(E)ᵢ—CO— in the general formula (2) include terephthalic acid, isophthalic acid, diphenyl ether dicarboxylic acid, bis (carboxyphenyl) hexafluoropropane, biphenyldicarboxylic acid, benzophenonedicarboxylic acid, and triphenyldicarboxylic acid. Examples of a tricarboxylic acid as the acid component include trimellitic acid, trimesic acid, diphenyl ether tricarboxylic acid, and biphenyltricarboxylic acid. Examples of a tetracarboxylic acid as the acid component include aromatic tetracarboxylic acids such as pyromellitic acid, 3,3′,4,4′-biphenyltetracarboxylic acid, 2,3,3′,4′-biphenyltetracarboxylic acid, 2,2′,3,3′-biphenyltetracarboxylic acid, 3,3′,4,4′-benzophenonetetracarboxylic acid, 2,2′,3,3′-benzophenonetetracarboxylic acid, 2,2-bis(3,4-dicaroxyphenyl)hexafluoropropane, 2,2-bis(2,3-dicaroxyphenyl)hexafluoropropane, 1,1-bis(3,4-dicarboxyphenyl)ethane, 1,1-bis(2,3-dicarboxyphenyl)ethane, bis(3,4-dicarboxyphenyl)methane, bis(2,3-dicarboxyphenyl)methane, bis(3,4-dicarboxyphenyl)sulfone, bis(3,4-dicarboxyphenyl) ether, 1,2,5,6-naphthalenetetracarboxylic acid, 2,3,6,7-naphthalenetetracarboxylic acid, 2,3,5,6-pyridinetetracarboxylic acid, and 3,4,9,10-perylenetetracarboxylic acid; aliphatic tetracarboxylic acids such as butanetetracarboxylic acid, and 1,2,3,4-cyclopentanetetracarboxylic acid.

In each of tricarboxylic acids and tetracarboxylic acids out of these examples, one or two carboxyl groups thereof correspond to the group E in the general formula (2). It is more preferred to use a compound obtained by substituting any one of the dicarboxylic acids, tricarboxylic acids and tetracarboxylic acids given above as the examples with, e.g., one to four of the groups E in the general formula (2), preferably with one to four hydroxyl groups, sulfonate groups, sulfonamide groups, or sulfonic acid ester groups. These acids may be used singly or in combination of two or more thereof in the form of the acid as it is, or in the form of one or more acid anhydrides or active esters.

In the general formula (2), E represents a group selected from OR⁴, SO₃R⁴, CONR⁴R⁵, COOR⁴, and SO₂NR⁴R⁵. R⁴ and R⁵ each represent a hydrogen atom or a monovalent hydrocarbon group having 1 to 20 carbon atoms. E is more preferably a hydroxyl group from the viewpoint of the solubility of the heat-resistant resin precursor in alkaline developers. In the general formula (2), i represents an integer of 0 to 4.

In the general formula (2), A represents a structural component of a diamine, and represents a structure represented by the general formula (4):

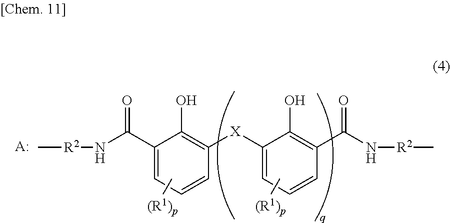

In the general formula (4), each R¹ represents an alkyl group having 1 to 5 carbon atoms; each p represents an integer of 0 to 2; and q represents an integer of 0 to 100. Each R² represents a bivalent aliphatic, alicyclic, or aromatic group, a bivalent organic group in which plural aromatic groups are bonded to each other through a single bond, a bivalent organic group in which plural aromatic groups are bonded to each other through —O—, —CO—, —SO₂—, —CH₂, —C(CH₃)₂—, or —C(CF₃)₂— wherein each F is fluorine. X represents —O—, —S—, —CO—, —SO₂—, —CH₂, —C(CH₃)₂—, —C(CH₃) (C₂H₅)—, or —C(CF₃)₂— wherein each F is fluorine.

The heat-resistant resin precursor may have, instead of A, a structural unit having any other diamine residue as far as the precursor is a precursor including the structural unit represented by the general formula (2) as a main component. Examples of the other diamine residue include respective residues of the following: hydroxyl-group-containing diamines such as bis(3-amino-4-hydroxyphenyl)hexafluoropropane (BAHF), bis(3-amino-4-hydroxyphenyl)sulfone, bis(3-amino-4-hydroxyphenyl)propane, bis(3-amino-4-hydroxyphenyl)methylene, bis(3-amino-4-hydroxyphenyl) ether, bis(3-amino-4-hydroxy)biphenyl, and bis(3-amino-4-hydroxyphenyl)fluorene; carboxyl-group-containing diamines such as 3, 5-diaminobenzoic acid, and 3-carboxyl-4,4′-diaminodiphenyl ether; sulfonic-acid-containing diamines such as 3-sulfonic acid-4,4′-diaminodiphenyl ether; and dithiohydroxyphenylenediamine.

Other examples thereof include respective residues of the following: 3,4′-diaminodiphenyl ether, 4,4′-diaminodiphenyl ether, 3,4′-diaminodiphenylmethane, 4,4′-diaminodiphenylmethane, 3,4′-diaminodiphenylsulfone, 4,4′-diaminodiphenylsulfone, 3,4′-diaminodiphenyl sulfide, 4,4′-diaminodiphenyl sulfide, 1,4-bis(4-aminophenoxy)benzene, benzine, m-phenylenediamine, p-phenylenediamine, 1,5-naphthalenediamine, 2,6-naphthalenediamine, bis(4-aminophenoxyphenyl)sulfone, bis(3-aminophenoxyphenyl)sulfone, bis(4-aminophenoxy)biphenyl, bis{4-(4-aminophenoxy)phenyl} ether, 1,4-bis(4-aminophenoxy)benzene, 2,2′-dimethyl-4,4′-diaminobiphenyl, 2,2′-diethyl-4,4′-diaminobiphenyl, 3,3′-dimethyl-4,4′-diaminobiphenyl, 3,3′-diethyl-4,4′-diaminobiphenyl, 2,2′,3,3′-tetramethyl-4,4′-diaminobiphenyl, 3,3′,4,4′-tetramethyl-4,4′-diaminobiphenyl, and 2,2′-di(trifluoromethyl)-4,4′-diaminobiphenyl; and compounds each obtained by substituting an aromatic ring of any one of these examples with an alkyl group or a halogen atom.

Moreover, it is allowable to use a residue of, e.g., an aliphatic cyclohexyldiamine or methylenebiscyclohexylamine. These diamines may be substituted with, e.g., a carbon group such as a methyl or ethyl group, or a radical of F, Cl, Br or I. These diamines may be used singly or in combination of two or more thereof in the form of one or more diamines, or in the form of the corresponding diisocyanate and trimethylsilylated diamine. A residue of any aromatic diamine is preferred from the viewpoint of the thermal resistance of the resultant polymer.

### <Heat-Resistant Resin Including Structural Unit Represented by General Formula (3) as Main Component>

The following will describe a heat-resistant resin including the structural unit represented by the general formula (3) as a main component.

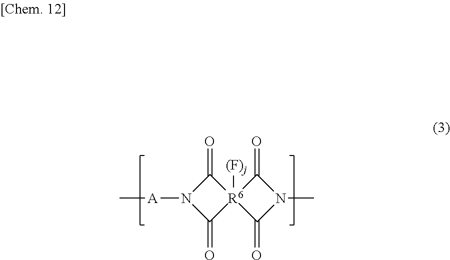

In the present invention, the resin represented by the general formula (3) denotes a resin having a polyimide structure. In the general formula (3), R⁶ represents a tetravalent to octavalent organic group having 2 to 30 carbon atoms, and is preferably an organic group having 1 or 2 aromatic rings. More preferred examples of the structure R⁶ in the general formula (2) are structures illustrated below, or any structure in which any one of these structures is partially substituted with one to four groups or atoms out of alkyl groups each having 1 to 20 carbon atoms, fluoroalkyl groups, alkoxyl groups, ester groups, nitro groups, cyano groups, fluorine atoms, and chlorine atoms.

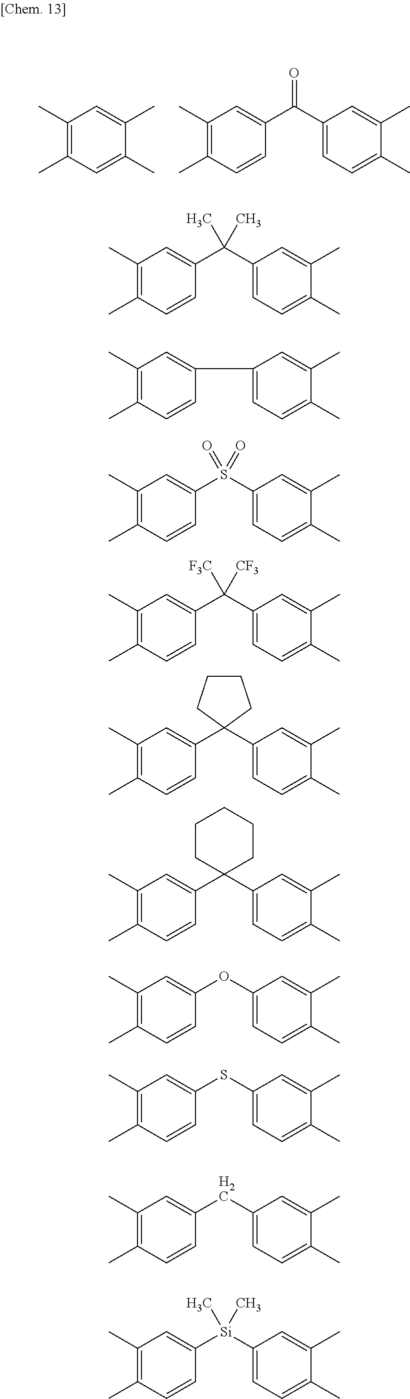

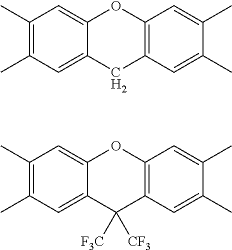

In the general formula (3), F represents a group selected from OR⁷, SO₃R⁷, CONR⁷R⁸, COOR⁷, and SO₂NR⁷R⁸. R⁷ and R⁸ each independently represent a hydrogen atom or a monovalent hydrocarbon group having 1 to 10 carbon atoms. Out of these groups, F is preferably a hydroxyl, carboxyl, ester, sulfonate, sulfonamide, or sulfonic acid ester group from the viewpoint of the solubility of the heat-resistant resin into alkaline developers. In the general formula (3), j represents an integer of 0 to 4.

In the general formula (3), A represents a structural component of a diamine, and represents a structure represented by the general formula (4):

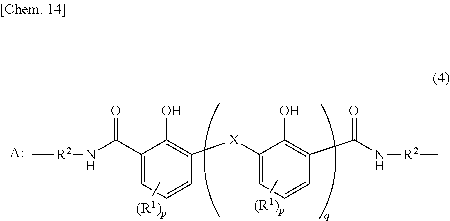

In the general formula (4), each R¹ represents an alkyl group having 1 to 5 carbon atoms; each p represents an integer of 0 to 2; and q represents an integer of 0 to 100. Each R² represents any case of a bivalent aliphatic, alicyclic, or aromatic group, any case of a bivalent organic group in which plural aromatic groups are bonded to each other through a single bond, or any case of a bivalent organic group in which plural aromatic groups are bonded to each other through —O—, —CO—, —SO₂—, —CH₂—, —C(CH₃)₂—, or —C(CF₃)₂— wherein each F is fluorine. X represents —O—, —S—, —CO—, —SO₂—, —CH₂—, —C(CH₃)₂—, —C(CH₃) (C₂H₅)—, or —C(CF₃)₂— wherein each F is fluorine.

The heat-resistant resin may have, instead of A, a structural unit having any other diamine residue as far as the resin is a resin including the structural unit represented by the general formula (3) as a main component.

Examples of the other diamine residue include structures illustrated below, and any structure in which any one of these structures is partially substituted with one to four groups or atoms out of alkyl groups each having 1 to 20 carbon atoms, fluoroalkyl groups, alkoxyl groups, ester groups, nitro groups, cyano groups, fluorine atoms, and chlorine atoms.

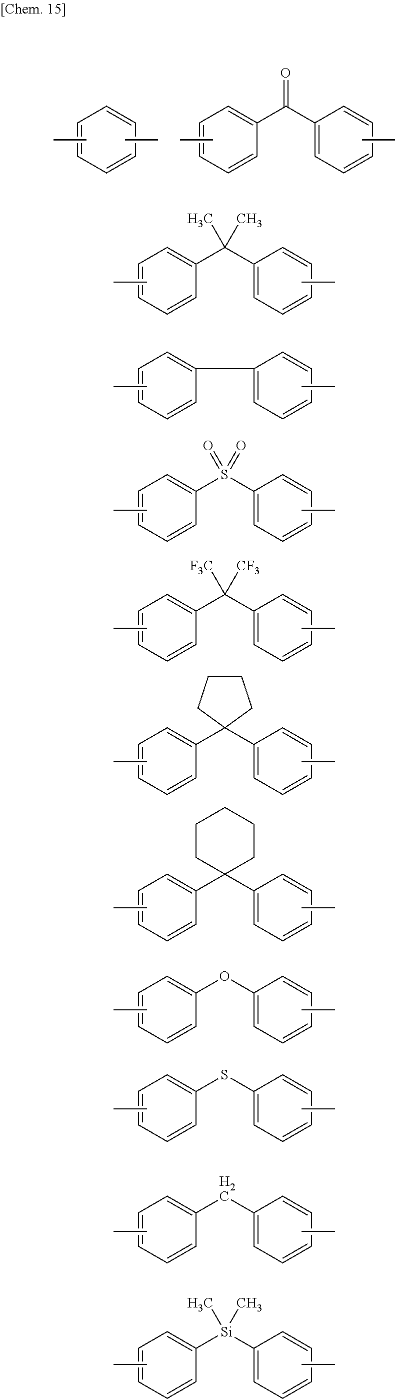

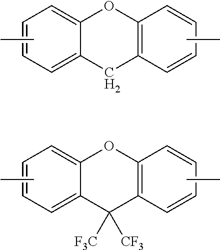

Examples of the other diamine residue include the following structures:

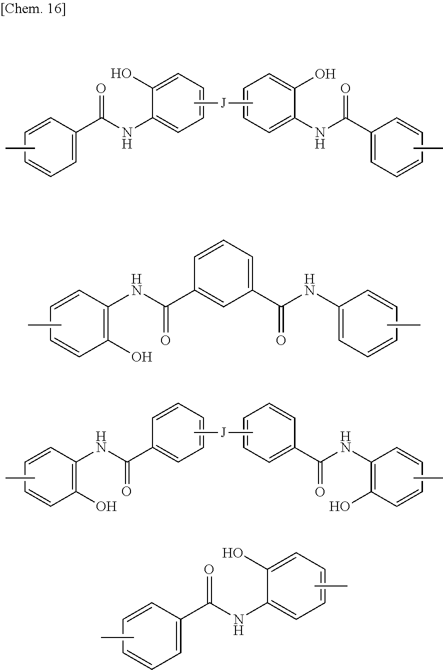

In these formulae, each J represents a direct bond, —COO—, —CONH—, —CH₂—, C₂H₄—, —O—, —C₃H₆—, —SO₂—, —S—, —Si(CH₃)₂—, —O—Si(CH₃)₂—O—, —C₆H₄—, —C₆H₄—O—C₆H₄—, —C₆H₄—C₃H₆C₆H₄—, or —C₆H₄—C₃F₆—C₆H₄—.

### <Heat-Resistant Resin Including Structural Unit Represented by General Formula (5) as Main Component>

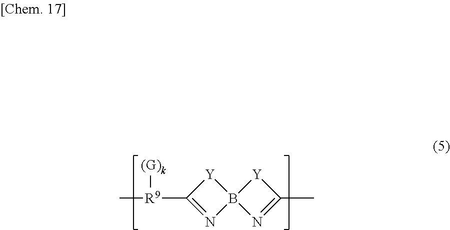

In the present invention, the resin represented by the general formula (5) denotes a resin having a cyclic structure such as a polybenzoxazole, a polybenzimidazole, or a polybenzothiazole.

In the general formula (5), R⁹ represents a bivalent to hexavalent organic group having 2 to 30 carbon atoms, and is preferably an organic group having 1 to 4 aromatic rings. In the general formula (5), preferred examples of the structure R⁹-(G)ₖ include structures illustrated below, and any structure in which any one of these structures is partially substituted with one to four groups or atoms out of alkyl groups each having 1 to 20 carbon atoms, fluoroalkyl groups, alkoxyl groups, ester groups, nitro groups, cyano groups, fluorine atoms, and chlorine atoms.

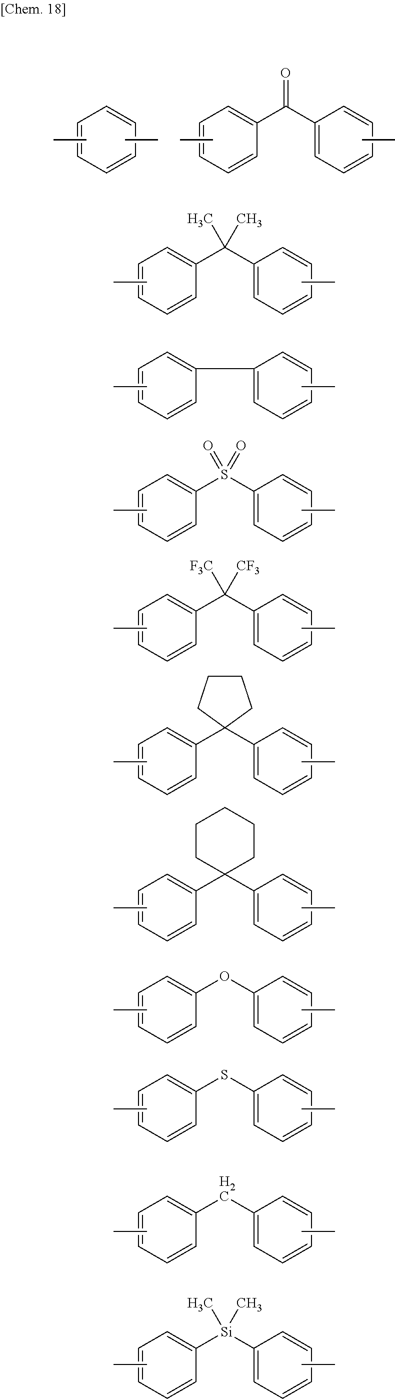

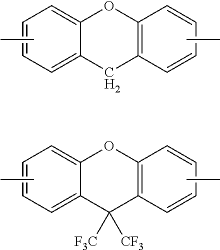

Examples of the structure R⁹-(G)ₖ also include the following structures:

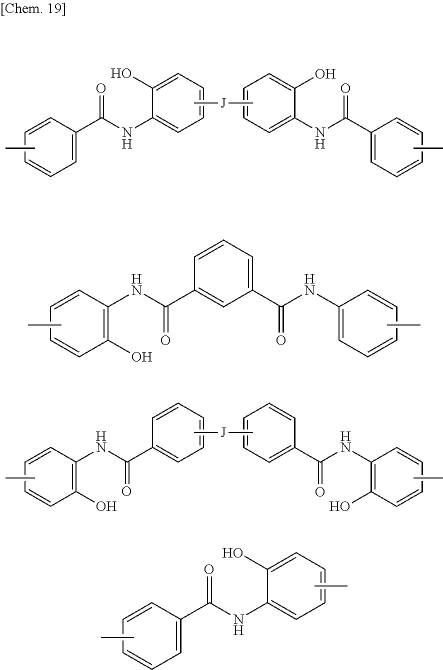

In these formulae, each J represents a direct bond, —COO—, —CONH—, —CH₂—, —C₂H₄—, —O—, —C₃H₆—, —SO₂—, —S—, —Si (CH₃)₂—, —O—Si(CH₃)₂—O—, —C₆H₄—, —C₆H₄—O—C₆H₄—, —C₆H₄—C₃H₆—C₆H₄—, or —C₆H₄—C₃F₆—C₆H₄—.

In the formula (5), G represents a group selected from OR¹⁰, SO₃R¹⁰, CONR¹⁰R¹¹, COOR¹⁰, and SO₂NR¹⁰R¹¹. R¹⁰ and R¹¹ each independently represent a hydrogen atom or a monovalent hydrocarbon group having 1 to 10 carbon atoms. Out of these groups, G is preferably a hydroxyl, carboxyl, ester, sulfonate, sulfonamide, or sulfonic acid ester group from the viewpoint of the solubility of the heat-resistant resin into alkaline developers. In the general formula (5), k represents an integer of 0 to 4.

In the general formula (5), B represents a structural component of a diamine, and each represent a structure represented by the general formula (6):

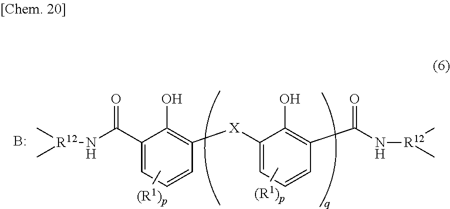

In the general formula (6), each R¹ represents an alkyl group having 1 to 5 carbon atoms; each p represents an integer of 0 to 2; q represents an integer of 0 to 100; each R¹² represents any case of a trivalent aliphatic group, alicyclic group, or aromatic group, any case of a trivalent organic group in which plural aromatic groups are bonded to each other through a single bond, or any case of a trivalent organic group in which plural aromatic groups are bonded to each other through —O—, —CO—, —SO₂—, —CH₂—, —C(CH₃)₂—, or —C(CF₃)₂— wherein each F is fluorine; and X represents —O—, —S—, —CO—, —SO₂—, —CH₂—, —C(CH₃)₂—, —C(CH₃) (C₂H₅)—, or —C(CF₃)₂—.

The heat-resistant resin may have, instead of B, a structural unit having any other diamine residue as far as the resin includes the structural unit represented by the general formula (5) as a main component.

Examples of the other diamine residue include structures illustrated below, and any structure in which any one of these structures is partially substituted with one to four groups or atoms out of alkyl groups each having 1 to 20 carbon atoms, fluoroalkyl groups, alkoxyl groups, ester groups, nitro groups, cyano groups, fluorine atoms, and chlorine atoms.

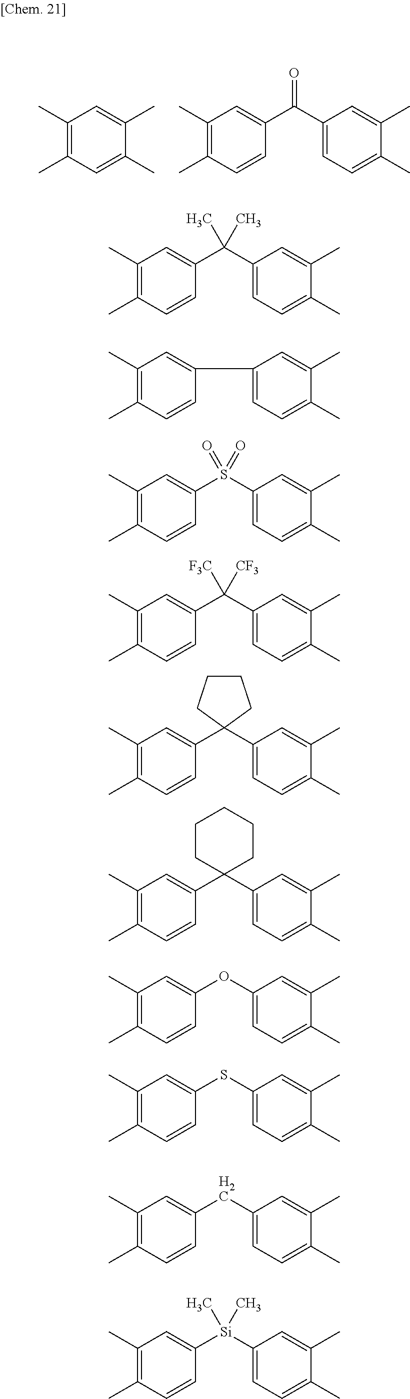

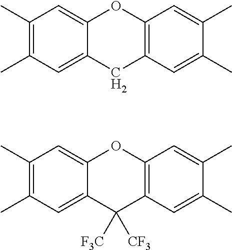

In the general formula (5), each Y is selected from NH, O and S.

In the heat-resistant resin precursor of the present invention, the recurring number of its structural units ranges preferably from 10 to 100,000. When the recurring number is from 10 to 100,000, the precursor gains an appropriate solubility in alkaline developers, and a heat-resistant resin obtained after the thermal treatment of the precursor gains a good elongation. From the viewpoint of the solubility of the heat-resistant resin precursor in alkaline developers, the recurring number is preferably 1,000 or less, more preferably 100 or less. From the viewpoint of an improvement in the elongation, the recurring number is preferably 20 or more.

About the heat-resistant resin precursor of the present invention, from the viewpoint of the solubility of the precursor in alkaline developers, the weight-average molecular weight (hereinafter referred to as Mw) thereof is preferably 100,000 or less, more preferably 50,000 or less. From the viewpoint of an improvement in the elongation, the Mw is preferably 10, 000 or more, more preferably 20,000 or more. The number-average molecular weight (hereinafter referred to as Mn) thereof is preferably 50,000 or less, more preferably 30,000 or less. The Mn is preferably 3,000 or more, more preferably 5,000 or more.

The weight-average molecular weight Mw and the number-average molecular weight Mn of the heat-resistant resin precursor are each easily measurable in terms of that of polystyrene by, for example, gel permeation chromatography (GPC), a light scattering method, or a small angle X-ray scattering technique. The recurring number n of the structural units of the heat-resistant resin precursor is as follows: n=Mw/M in which the molecular weight of each of the structural units is M, and the weight-average molecular weight of the polymer is Mw. In the present invention, the recurring number n denotes a value calculated out using GPC measurement in terms of that of polystyrene, which is the simplest and easiest method.

In order to improve in adhesiveness onto a substrate, it is allowable to copolymerize, into the heat-resistant resin precursor, an aliphatic group having a siloxane structure as far as the thermal resistance of the resin is not lowered. The precursor is specifically a resin into which a diamine component as described in the following is copolymerized in a proportion of 1 to 10% by mole: for example, 3-bis(3-aminopropyl)tetramethyldisiloxane (SiDA), or bis(p-amino-phenyl)octamethylpentasiloxane.

When terminals of such a resin are blocked with a monoamine having a functional group selected from the group consisting of hydroxyl, carboxyl, sulfonate, and thiol groups, the solution velocity of the resin in aqueous alkaline solution can be adjusted into a preferred range.

Examples of the monoamine include aniline, naphthylamine, amino pyridine, and the following compounds each having a phenolic hydroxyl group: 3-amino-4,6-dihydroxypyrimidine, 2-aminophenol, 3-aminophenol, 4-aminophenol, 5-amino-8-hydroxyquinoline, 4-amino-8-hydroxyquinoline, 1-hydroxy-8-aminonaphthalene, 1-hydroxy-7-aminonaphthalene, 1-hydroxy-6-aminonaphthalene, 1-hydroxy-5-aminonaphthalene, 1-hydroxy-4-aminonaphthalene, 1-hydroxy-3-aminonaphthalene, 1-hydroxy-2-aminonaphthalene, 1-amino-7-hydroxynaphthalene, 2-hydroxy-7-aminonaphthalene, 2-hydroxy-6-aminonaphthalene, 2-hydroxy-5-aminonaphthalene, 2-hydroxy-4-aminonaphthalene, 2-hydroxy-3-aminonaphthalene, and 1-amino-2-hydroxynaphthalene. Other examples thereof include monoamines each having a carboxyl group, such as 1-carboxy-8-aminonaphthalene, 1-carboxy-7-aminonaphthalene, 1-carboxy-6-aminonaphthalene, 1-carboxy-5-aminonaphthalene, 1-carboxy-4-aminonaphthalene, 1-carboxy-3-aminonaphthalene, 1-carboxy-2-aminonaphthalene, 1-amino-7-carboxynaphthalene, 2-carboxy-7-aminonaphthalene, 2-carboxy-6-aminonaphthalene, 2-carboxy-5-aminonaphthalene, 2-carboxy-4-aminonaphthalene, 2-carboxy-3-aminonaphthalene, 1-amino-2-carboxynaphthalene, 2-aminonicotinic acid, 4-aminonicotinic acid, 5-aminonicotinic acid, 6-aminonicotinic acid, 4-aminosalicylic acid, 5-aminosalicylic acid, 6-aminosalicylic acid, 3-amino-o-toluic acid, ameride, 2-aminobenzoic acid, 3-aminobenzoic acid, and 4-aminobenzoic acid; 2-aminobenzenesulfonic acid, 3-aminobenzenesulfonic acid, and 4-aminobenzenesulfonic acid; monoamines each having a thiol group, such as 5-amino-8-mercaptoquinoline, 4-amino-8-mercaptoquinoline, 1-mercapto-8-aminonaphthalene, 1-mercapto-7-aminonaphthalene, 1-mercapto-6-aminonaphthalene, 1-mercapto-5-aminonaphthalene, 1-mercapto-4-aminonaphthalene, 1-mercapto-3-aminonaphthalene, 1-mercapto-2-aminonaphthalene, 1-amino-7-mercaptonaphthalene, 2-mercapto-7-aminonaphthalene, 2-mercapto-6-aminonaphthalene, 2-mercapto-5-aminonaphthalene, 2-mercapto-4-aminonaphthalene, 2-mercapto-3-aminonaphthalene, 1-amino-2-mercaptonaphthalene, 3-amino-4,6-dimercaptopyrimidine, 2-aminothiophenol, 3-aminothiophenol, and 4-aminothiophenol.

Out of these examples, preferred are 5-amino-8-hydroxyquinoline, 1-hydroxy-7-aminonaphthalene, 1-hydroxy-6-aminonaphthalene, 1-hydroxy-5-aminonaphthalene, 1-hydroxy-4-aminonaphthalene, 2-hydroxy-7-aminonaphthalene, 2-hydroxy-6-aminonaphthalene, 2-hydroxy-5-aminonaphthalene, 1-carboxy-7-aminonaphthalene, 1-carboxy-6-aminonaphthalene, 1-carboxy-5-aminonaphthalene, 2-carboxy-7-aminonaphthalene, 2-carboxy-6-aminonaphthalene, 2-carboxy-5-aminonaphthalene, 2-aminobenzoic acid, 3-aminobenzoic acid, 4-aminobenzoic acid, 4-aminosalicylic acid, 5-aminosalicylic acid, 6-aminosalicylic acid, 2-aminobenzenesulfonic acid, 3-aminobenzenesulfonic acid, 4-aminobenzenesulfonic acid, 3-amino-4,6-dihydroxypyrimidine, 2-aminophenol, 3-aminophenol, 4-aminophenol, 2-aminothiophenol, 3-aminothiophenol, and 4-aminothiophenol since these each have a hydrophilic group. These monoamines may be used singly or in combination of two or more thereof.

When terminals of the resin are blocked with an acid anhydride, acid chloride or monocarboxylic acid, the dissolution rate of the resin in aqueous alkaline solution can be adjusted into a preferred range.

Examples of the acid anhydride, acid chloride or monocarboxylic acid include acid anhydrides such as phthalic anhydride, maleic anhydride, nadic acid, cyclohexanedicarboxylic anhydride, and 3-hydroxylphthalic anhydride; monocarboxylic acids such as 2-carboxyphenol, 3-carboxyphenol, 4-carboxyphenol, 2-carboxythiophenol, 3-carboxythiophenol, 4-carboxythiophenol, 1-hydroxy-8-carboxynaphthalene, 1-hydroxy-7-carboxynaphthalene, 1-hydroxy-6-carboxynaphthalene, 1-hydroxy-5-carboxynaphthalene, 1-hydroxy-4-carboxynaphthalene, 1-hydroxy-3-carboxynaphthalene, 1-hydroxy-2-carboxynaphthalene, 1-mercapto-8-carboxynaphthalene, 1-mercapto-7-carboxynaphthalene, 1-mercapto-6-carboxynaphthalene, 1-mercapto-5-carboxynaphthalene, 1-mercapto-4-carboxynaphthalene, 1-mercapto-3-carboxynaphthalene, 1-mercapto-2-carboxynaphthalene, 2-carboxybenzenesulfonic acid, 3-carboxybenzenesulfonic acid and 4-carboxybenzenesulfonic acid, and monoacid chloride compounds each obtained by chlorinating a carboxyl group of any one of these carboxylic acids; mono acid chloride compounds each obtained by chlorinating only a monocarboxyl group of any one of the following dicarboxylic acids: terephthalic acid, phthalic acid, maleic acid, cyclohexane dicarboxylic acid, 3-hydroxyphthalic acid, 5-norbornene-2,3-dicarboxylic acid, 1,2-dicarboxynaphthalene, 1,3-dicarboxynaphthalene, 1,4-dicarboxynaphthalene, 1,5-dicarboxynaphthalene, 1,6-dicarboxynaphthalene, 1,7-dicarboxynaphthalene, 1,8-dicarboxynaphthalene, 2,3-dicarboxynaphthalene, 2,6-dicarboxynaphthalene, and 2,7-dicarboxynaphthalene; and active ester compounds each obtained by causing a monoacid chloride compound to react with N-hydroxybenzotriazole, or N-hydroxy-5-norbornene-2,3-dicarboxyimide.

Out of these examples, preferred are acid anhydrides such as phthalic anhydride, maleic anhydride, nadic acid, cyclohexanedicarboxylic anhydride, and 3-hydroxylphthalic anhydride; monocarboxylic acids such as 3-carboxyphenol, 4-carboxyphenol, 3-carboxythiophenol, 4-carboxythiophenol, 1-hydroxy-7-carboxynaphthalene, 1-hydroxy-6-carboxynaphthalene, 1-hydroxy-5-carboxynaphthalene, 1-mercapto-7-carboxynaphthalene, 1-mercapto-6-carboxynaphthalene, 1-mercapto-5-carboxynaphthalene, 3-carboxybenzenesulfonic acid and 4-carboxybenzenesulfonic acid, and monoacid chloride compounds each obtained by chlorinating a carboxyl group of any one of these carboxylic acids; mono acid chloride compounds each obtained by chlorinating only a monocarboxyl group of any one of terephthalic acid, phthalic acid, maleic acid, cyclohexane dicarboxylic acid, 1,5-dicarboxynaphthalene, 1,6-dicarboxynaphthalene, 1,7-dicarboxynaphthalene, and 2,6-dicarboxynaphthalene; and active ester compounds each obtained by causing a mono acid chloride compound to react with N-hydroxybenzotriazole, or N-hydroxy-5-norbornene-2,3-dicarboxyimide. These may be used singly or in combination of two or more thereof.

The content of the terminal blocking agent, such as the monoamine, acid anhydride, acid chloride or monocarboxylic acid, is preferably from 0.1 to 60% by mole of the whole of the resin, in particular preferably from 5 to 50% by mole thereof. When the content is set into such a range, a resin composition can be yielded which gives an appropriate viscosity of a solution used when coated, and which has excellent film properties.

The terminal blocking agent introduced into the resin can be easily detected by the following method: for example, the resin into which the terminal blocking agent is introduced is dissolved into an acidic solution to decompose the resin into an amine component and an acid anhydride component which are constituent units of the resin; and then the resultant is measured by gas chromatography (GC) or NMR. In this way, the terminal blocking agent can be easily detected. Separately from this method, the resin into which the terminal blocking agent is introduced can be directly detected by pyrolysis gas chromatography (PGC), or infrared spectrum and ¹³C-NMR spectrum measurements.

<Photosensitive Compound (b)>

The photosensitive resin composition of the present invention includes a photosensitive compound (b). The photosensitive compound (b) may be a quinonediazide compound (b1) or a photopolymerization initiator (b2). The quinonediazide compound (b1) is a compound which is irradiated with light to generate an acid, thereby increasing a property in solubility in aqueous alkaline solution of the light-irradiated portions. The photopolymerization initiator (b2) denotes a compound which is exposed to light to undergo bond-cleavage and/or reaction, thereby generating radicals.

When the composition includes the quinonediazide compounds (b1), an acid is generated in the light-irradiated portions so that the light-irradiated portions are increased in solubility in aqueous alkaline solution. Consequently, a positive relief pattern can be yielded in which the light-irradiated portions are dissolved. Moreover, when the composition includes the quinonediazide compounds (b1), and an epoxy compound or a thermally crosslinking agent which will be detailed later, an acid generated in the light-irradiated portions promotes a crosslinking reaction of the epoxy compound or the thermally crosslinking agent. Consequently, a negative relief pattern can be yielded in which the light-irradiated portions are undissolved. Furthermore, when the resin composition includes the photopolymerization initiator (b2) and a radical polymerizable compound which will be detailed later, radical polymerization advances so that light-exposed portions of a film of the resin composition are undissolved in an alkaline developer. Consequently, a negative pattern can be formed.

The quinonediazide compounds (b1) is, for example, a compound in which a sulfonic acid of a quinonediazide is ester-bonded to a polyhydroxy compound, a compound in which a sulfonic acid of a quinonediazide is sulfonamide-bonded to a polyamino compound, or a compound in which a sulfonic acid of a quinonediazide is ester-bonded and/or sulfonamide-bonded to a polyhydroxypolyamine compound. In order to improve contrast between the light-exposed portions and light-unexposed portions, 50% or more by mole of the whole of functional groups of the polyhydroxy compound or polyamino compound is preferably substituted with a quinonediazide. The use of such a quinonediazide compound makes it possible to yield a positive photosensitive resin composition photosensitive to an i-line (365 nm), an h-line (405 nm) and a g-line (436 nm) from a mercury lamp, which are ordinary ultraviolet rays.

Examples of the polyhydroxy compound include Bis-Z, Bis P-EZ, TekP-4HBPA, Tris P-HAP, Tris P-PA, Tris P-SA, Tris OCR-PA, Bis OCHP-Z, Bis P-MZ, Bis P-PZ, Bis P-IPZ, Bis OCP-IPZ, Bis P-CP, Bis RS-2P, Bis RS-3P, Bis P-OCHP, Methylene tris-FR-CR, Bis RS-26X, DML-MBPC, DML-MBOC, DML-OCHP, DML-PCHP, DML-PC, DML-PTBP, DML-34X, DML-EP, DML-POP, Dimethylol-Bis OC-P, DML-PFP, DML-PSBP, DML-M Tris PC, TriML-P, Tri ML-35XL, TML-BP, TML-HQ, TML-pp-BPF, TML-BPA, TMOM-BP, HML-TPPHBA, and HML-TPHAP ((each showing a trade name) manufactured by Honshu Chemical Industry Co., Ltd.); BIR-OC, BIP-PC, BIR-PC, BIR-PTBP, BIR-PCHP, BIP-BIOC-F, 4PC, BIR-BIPC-F, TEP-BIP-A, 46DMOC, 46DMOEP, and TM-BIP-A ((each showing a trade name) manufactured by Asahi Organic Chemicals Industry Co., Ltd.); and 2,6-dimethoxymethyl-4-t-butylphenol, 2,6-dimethoxymethyl-p-cresol, 2,6-diacetoxymethyl-p-cresol, naphthol, tetrahydroxybenzophenone, methyl gallate, bisphenol A, bisphenol E, methylenebisphenol, and Bis P-AP ((showing a trade name), manufactured by Honshu Chemical Industry Co., Ltd.). These are commercially available. However, the polyhydroxy compound is not limited to these examples.

Examples of the polyamino compound include 1,4-phenylenediamine, 1,3-phenylenediamine, 4,4′-diaminodiphenyl ether, 4,4′-diaminodiphenylmethane, 4,4′-diaminodiphenylsulfone, and 4,4′-diaminodiphenyl sulfide. However, the polyamino compound is not limited to these examples.

Examples of the polyhydroxypolyamino compound include 2,2-bis(3-amino-4-hydroxyphenyl)hexafluoropropane, and 3,3′-dihydroxybenzidine. However, the polyhydroxypolyamino compound is not limited to these examples.

In the present invention, as the quinonediazide, any one of the following is preferably used: 5-naphthoquinonediazidesulfonyl group, and 4-naphthoquinonediazidesulfonyl group. Usable is also a naphthoquinonediazidesulfonyl ester compound, which has in a single molecule a 4-naphthoquinonediazidesulfony group and a 5-naphthoquinonediazidesulfony group. Usable are a 4-naphthoquinonediazidesulfony ester compound and a 5-naphthoquinonediazidesulfony ester compound in combination.

The molecular weight of the quinonediazide compound is preferably 1500 or less, more preferably 1200 or less. When the molecular weight is 1500 or less, the quinonediazide compound is sufficiently thermally decomposed in a thermal treatment of the resin composition after the composition is patterned, so that a cured film can be yielded which is excellent in thermal resistance, mechanical properties and adhesiveness. The molecular weight of the quinonediazide compound is preferably 300 or more, more preferably 350 or more.

The content of the quinonediazide compounds (b1) is preferably 1 part or more by mass, more preferably 3 parts or more by mass for 100 parts by mass of the heat-resistant resin or heat-resistant resin precursor (a). The content is also preferably 50 parts or less by mass, more preferably 40 parts or less by mass. When the content is set into such a range, a resin composition processable into a pattern with a good sensitivity can be yielded.

A method for synthesizing the quinonediazide compound used in the present invention is, for example, a method of causing a phenolic compound to react with 5-naphthoquinonediazidesulfonyl chloride in the presence of triethylamine. A method for synthesizing the phenolic compound is, for example, a method of causing an α-(hydroxyphenyl) styrene derivative to react with a polyhydric phenolic compound.

The photopolymerization initiator (b2) is preferably, for example, a benzyl ketal photopolymerization initiator, an α-hydroxyketone photopolymerization initiator, an α-aminoketone photopolymerization initiator, an acylphosphine oxide photopolymerization initiator, an oxime ester photopolymerization initiator, an acridine photopolymerization initiator, a titanocene photopolymerization initiator, a benzophenone photopolymerization initiator, an acetophenone photopolymerization initiator, an aromatic ketoester photopolymerization initiator, or a benzoic acid ester photopolymerization initiator. From the viewpoint of an improvement of the resin composition in sensitivity when the composition is exposed to light, more preferred is the α-hydroxyketone photopolymerization initiator, α-aminoketone photopolymerization initiator, acylphosphine oxide photopolymerization initiator, oxime ester photopolymerization initiator, acridine photopolymerization initiator, or benzophenone photopolymerization initiator. Even more preferred is the α-aminoketone photopolymerization initiator, acylphosphine oxide photopolymerization initiator or oxime ester photopolymerization initiator.

An example of the benzyl ketal photopolymerization initiator is 2,2-dimethoxy-1,2-diphenylethane-1-one.

Examples of the α-hydroxyketone photopolymerization initiator include 1-(4-isopropylphenyl)-2-hydroxy-2-methylpropane-1-one, 2-hydroxy-2-methyl-1-phenylpropane-1-one, 1-hydroxycyclohexyl phenyl ketone, 1-[4-(2-hydroxyethoxy)phenyl)]-2-hydroxy-2-methylpropane-1-one, and 2-hydroxy-1-[4-[4-(2-hydroxy-2-methylpropionyl)benzyl]pheny 1]-2-propane-1-one.

Examples of the α-aminoketone photopolymerization initiator include 2-methyl-1-[4-(methylthio)phenyl]-2-morpholinopropane-1-one, 2-benzyl-2-dimethylamino-1-(4-morpholinophenyl)-butane-1-one, 2-dimethylamino-2-(4-methylbenzyl)-1-(4-morpholinophenyl)-butane-1-one, and 3,6-bis(2-methyl-2-morpholinopropionyl)-9-octyl-9H-carbazole.

Examples of the acylphosphine oxide photopolymerization initiator include 2,4,6-trimethylbenzoyl-diphenylphosphine oxide, bis (2,4,6-trimethylbenzoyl)-phenylphosphine oxide, and bis (2,6-dimethoxybenzoyl)-(2,4,4-trimethylpentyl)phosphine oxide.

Examples of the oxime ester photopolymerization initiator include 1-phenypropane-1,2-dione-2-(O-ethoxycarbonyl)oxime, 1-phenyl butane-1,2-dione-2-(O-methoxycarbonyl)oxime, 1,3-diphenyl propane-1,2,3-trione-2-(O-ethoxycarbonyl)oxime, 1-[4-(phenylthio)phenyl]octane-1,2-dione-2-(O-benzoyl)oxime, 1-[4-[4-(carboxyphenyl)thio]phenyl]propane-1,2-dione-2-(O-acetyl)oxime, 1-[9-ethyl-6-(2-methylbenzoyl)-9H-carbazole-3-yl]ethanone-1-(O-acetyl)oxime, 1-[9-ethyl-6-(2-methyl-4-[1-(2,2-dimethyl-1,3-dioxolane-4-yl)methyloxy]benzoyl]-9H-carbazole-3-yl]ethanone-1-(O-acetyl) oxime, and “ADEKA ARKLS” ((registered trade name) NCI-831(manufactured by ADEKA CORPORATION).

An example of the acridine photopolymerization initiator is 1, 7-bis (acridine-9-yl)-n-heptane.

Examples of the titanocene photopolymerization initiator include bis(η5-2,4-cyclopentadiene-1-yl)-bis[2,6-difluoro)-3-(1H-py rrole-1-yl)phenyl]titanium (IV), and bis(η5-3-methyl-2,4-cyclopentadiene-1-yl)-bis(2,6-difluorop henyl)titanium (IV).

Examples of the benzophenone photopolymerization initiator include benzophenone, 4,4′-bis(dimethylamino)benzophenone, 4,4′-bis(diethylamino)benzophenone, 4-phenylbenzophenone, 4,4-dichlorobenzophenone, 4-hydroxybenzophenone, alkylated benzophenone, 3,3′,4,4′-tetrakis(t-butylperoxycarbonyl)benzophenone, 4-methylbenzophenone, dibenzyl ketone, and fluorenone. [0125]

Examples of the acetophenone photopolymerization initiator include 2,2-diethoxyacetophenone, 2,3-diethoxyacetophenone, 4-t-butyldichloroacetophenone, benzalacetophenone, and 4-azidebenzalacetophenone.

An example of the aromatic ketoester photopolymerization initiator is methyl 2-phenyl-2-oxyacetate.

Examples of the benzoic acid ester photopolymerization initiator include ethyl 4-dimethylaminobenzoate, (2-ethyl)hexyl 4-dimethylaminobenzoate, ethyl 4-diethylaminobenzoate, and methyl 2-benzoylbenzoate.

When the total amount of the heat-resistant resin or heat-resistant resin precursor (a), and a radical polymerizable compound (d), which will be detailed later, is regarded as 100 parts by mass in the present invention, the content of the photopolymerization initiator (b2) is preferably 0.1 parts or more by mass, more preferably 0.5 parts or more by mass, even more preferably 0.7 parts or more by mass, in particular preferably 1 part or more by mass. When the content is equal to or over the above-described preferred lower limit, the resin or precursor can be improved in sensitivity when exposed to light. In the meantime, the content is preferably 25 parts or less by mass, more preferably 20 parts or less by mass, even more preferably 17 parts or less by mass, in particular preferably 15 parts or less by mass. When the content is equal to or under the above-described preferred upper limit, the photosensitive resin composition can be improved in resolution after developed, and can further give a slightly tapered pattern shape.

<Solvent (c)>

The photosensitive resin composition of the present invention includes a solvent (c). Examples of the solvent include polar aprotic solvents such as N-methyl-2-pyrrolidone (NMP), γ-butyrolactone (GEL), N,N-dimethylformamide, N,N-dimethylacetoamide, dimethylsulfoxide, 1,3-dimethyl-2-imidazolidinone, N,N′ -dimethylpropyleneurea, N,N-dimethylisobutyric acid amide, and 3-methoxy-N,N-dimethylpropionamide; ethers such as tetrahydrofuran, dioxane, diethylene glycol ethyl methyl ether, and propylene glycol monomethyl ether; ketones such as acetone, methyl ethyl ketone, diisobutyl ketone, and diacetone alcohol;

esters such as ethyl acetate, propylene glycol monomethyl ether acetate, and ethyl lactate; and aromatic hydrocarbons such as toluene and xylene. In the present invention, these solvents may be used singly or in combination of two or more thereof. The content of the solvent is preferably 50 parts or more by mass, more preferably 100 parts or more by mass for 100 parts by mass of the heat-resistant resin or heat-resistant resin precursor (a). The content is also preferably 2000 parts or less by mass, more preferably 1500 parts or less by mass.

<Radical Polymerizable Compound (d)>

The photosensitive resin composition of the present invention may further include a radical polymerizable compound (d).

The radical polymerizable compound (d) denotes a compound having, in the molecule thereof, plural ethylenical double bonds. When the resin composition is exposed to light, the radical polymerization of the radical polymerizable compound (d) is advanced by radicals generated from the above-mentioned photopolymerization initiator (b2), so that exposed portions of a film of this composition are undissolved in an alkaline developer. Consequently, a negative pattern can be formed.

When the radical polymerizable compound (d) is incorporated into the resin composition, the composition is increasingly cured by UVs when exposed to the UVs, so as to be improvable in sensitivity at this exposure time. Additionally, after thermally cured, the composition is improved in crosslinkage density so that the resultant cured film can be improved in hardness.

The radical polymerizable compound (d) is preferably a compound having a (meth)acrylic group, which easily undergoes the advance of radical polymerization. From the viewpoint of an improvement in the sensitivity of the resin composition at the exposure time, and an improvement in the hardness of the cured film, the compound (d) is preferably a compound having, in the molecule thereof, two or more (meth) acrylic groups. The double bond equivalent of the radical polymerizable compound (d) is preferably from 80 to 400 g/mol from the viewpoint of an improvement in the sensitivity at the exposure time, and an improvement in the hardness of the cured film.

Examples of the radical polymerizable compound (d) include diethylene glycol di(meth)acrylate, triethylene glycol di(meth)acrylate, tetraethylene glycol di(meth)acrylate, propylene glycol di(meth)acrylate, trimethylolpropane di(meth)acrylate, trimethylolpropane tri(meth)acrylate, ethoxylated trimethylolpropane di(meth)acrylate, ethoxylated trimethylolpropane tri(meth)acrylate, ditrimethylolpropane tri(meth)acrylate, ditrimethylolpropane tetra(meth)acrylate, 1,3-butanediol di(meth)acrylate, neopentyl glycol di(meth)acrylate, 1,4-butanediol di(meth)acrylate, 1,6-hexanediol di(meth)acrylate, 1,9-nonanediol di(meth)acrylate, 1,10-decanediol di(meth)acrylate, dimethylol-tricyclodecane di(meth)acrylate, ethoxylated glycerin tri(meth)acrylate, pentaerythritol tri(meth)acrylate, pentaerythritol tetra(meth)acrylate, ethoxylated pentaerythritol tri(meth)acrylate, ethoxylated pentaerythritol tetra(meth)acrylate, dipentaerythritol penta(meth)acrylate, dipentaerythritol hexa(meth)acrylate, tripentaerythritol hepta(meth)acrylate, tripentaerythritol octa(meth)acrylate, tetrapentaerythritol nona(meth)acrylate, tetrapentaerythritol deca(meth)acrylate, pentapentaerythritol undeca(meth)acrylate, pentapentaerythritol dodeca(meth)acrylate, ethoxylated bisphenol A di(meth)acrylate, 2,2-bis[4-(3-(meth)acryloxy-2-hydroxypropoxy)phenyl]propane, 1,3,5-tris((meth)acryloxyethyl) isocyanurate, 1,3-bis((meth)acryloxyethyl) isocyanurate, 9,9-bis[4-(2-(meth)acryloxyethoxy)phenyl]fluorene, 9,9-bis[4-(3-(meth)acryloxypropoxy)phenyl]fluorene, and 9,9-bis(4-(meth)acryloxyphenyl)fluorene; and respective acid-modified products, ethylene-oxide-modified products, and propylene-oxide-modified products of these examples.

From the viewpoint of an improvement of the resin composition in sensitivity at the exposure, and an improvement in the hardness of the cured film, preferred are trimethylolpropane tri(meth)acrylate, ditrimethylolpropane tri (meth) acrylate, ditrimethylolpropane tetra (meth) acrylate, pentaerythritol tri(meth)acrylate, pentaerythritol tetra(meth)acrylate, dipentaerythritol penta(meth)acrylate, dipentaerythritol hexa(meth)acrylate, tripentaerythritol hepta(meth)acrylate, tripentaerythritol octa(meth)acrylate, 2,2-bis[4-(3-(meth)acryloxy-2-hydroxypropoxy)phenyl]propane, 1,3,5-tris((meth)acryloxyethyl) isocyanurate, 1,3-bis((meth)acryloxyethyl) isocyanurate, 9, 9-bis [4-(2-(meth) acryloxyethoxy) phenyl] fluorene, 9,9-bis[4-(3-(meth)acryloxypropoxy)phenyl]fluorene, and 9,9-bis(4-(meth)acryloxyphenyl)fluorene; and respective acid-modified products, ethylene-oxide-modified products, and propylene-oxide-modified products of these examples. From the viewpoint of an improvement in the resolution of the photosensitive composition that has been developed, more preferred are respective acid-modified products and ethylene-oxide-modified products.

From the viewpoint of an improvement in the resolution of the photosensitive composition that has been developed, preferred is also a compound obtained by causing a polybasic carboxylic acid or a polybasic carboxylic acid anhydride to react with a compound obtained by causing a compound having, in the molecule thereof, two or more glycidoxy groups, and an unsaturated carboxylic acid having an ethylenical double bond to undergo a ring-opening addition reaction.

When the total amount of the heat-resistant resin or heat-resistant resin precursor (a) and the radical polymerizable compound (d) is regarded as 100 parts by mass in the present invention, the content of the radical polymerizable compound (d) is preferably 15 parts or more by mass, more preferably 20 parts or more by mass, even more preferably 25 parts or more by mass, in particular preferably 30 parts or more by mass. The content which is equal to or over the above-described preferred lower limit makes it possible to improve the photosensitive resin composition in resolution when the composition is developed, and further give a slightly tapered pattern shape. In the meantime, the content is preferably 65 parts or less by mass, more preferably 60 parts or less by mass, even more preferably 55 parts or less by mass, in particular preferably 50 parts or less by mass. When the content is equal to or under the above-described preferred upper limit, the resultant cured film can be improved in thermal resistance, and can further give a slightly tapered pattern shape.

<Alkoxymethyl-Group-Containing Compound, and Cyclic-Polyether-Structure-Having Compound (e)>

The photosensitive resin composition of the present invention may include, besides the components (a) to (c), an alkoxymethyl-group-containing compound, and/or a cyclic-polyether-structure-having compound (e). The alkoxymethyl group and cyclic polyether structure each undergo crosslinking reaction in a temperature range of 150° C. or higher; thus, when the composition includes these compounds, the composition can be crosslinked by development followed by heating treatment that will be detailed later, so as to gain excellent mechanical properties.

The alkoxymethyl-group-containing compound is preferably a compound having two or more alkoxymethyl groups to raise the crosslinkage density of the composition. In order to raise the crosslinkage density and improve the mechanical properties, the compound is more preferably a compound having four or more alkoxymethyl groups. In the present invention, the alkoxymethyl-group-containing compound is preferably a compound having a group represented by a general formula (9) illustrated below, or a compound represented by a general formula (10) illustrated below. The compounds may be used together.

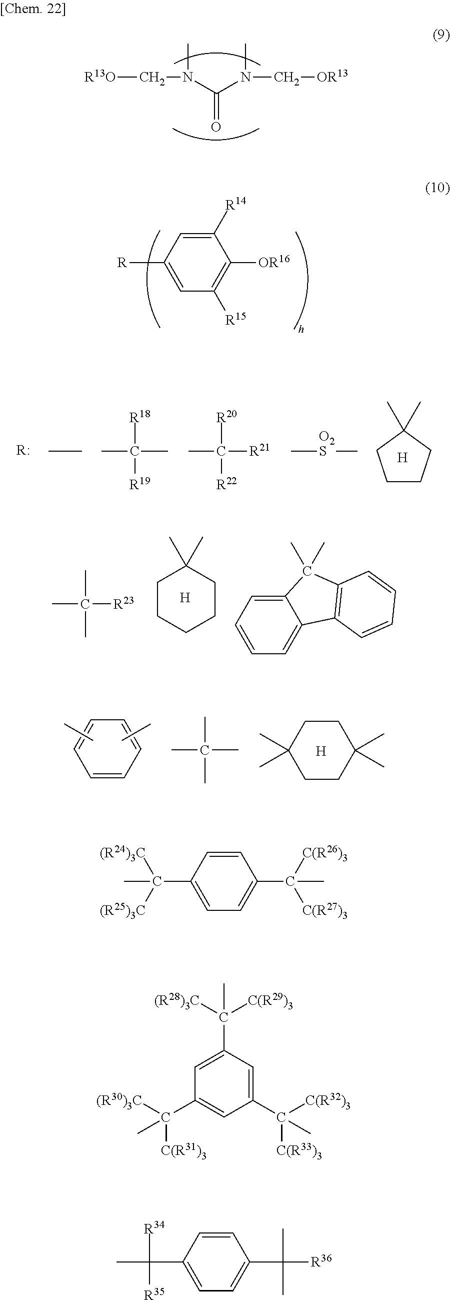

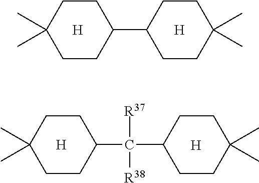

In the general formula (9), each R¹³ represents an alkyl group having 1 to 20 carbon atoms. From the viewpoint of the solubility of the compound of the formula (9) in the resin composition, each R¹³ is preferably an alkyl group having 1 to 10 carbon atoms, more preferably an alkyl group having 1 to 3 carbon atoms.

In the general formula (10), R¹⁴ and R¹⁵ each represent CH₂OR¹⁷. R¹⁷ represents an alkyl group having 1 to 6 carbon atoms. From the viewpoint of the solubility of the compound of the formula (10) in the resin composition, R¹⁴ and R¹⁵ are each more preferably an alkyl group having 1 to 3 carbon atoms. R¹⁶ represents a hydrogen atom, or a methyl or ethyl group. R¹⁸ to R³⁸ may be the same as or different from each other, and each represent a hydrogen atom, or an organic group having 1 to 20 carbon atoms. The symbol h represents an integer of 1 to 4.

Specific examples of the compound containing a group represented by the general formula (9) include compounds illustrated below. However, the compound is not limited to these examples.

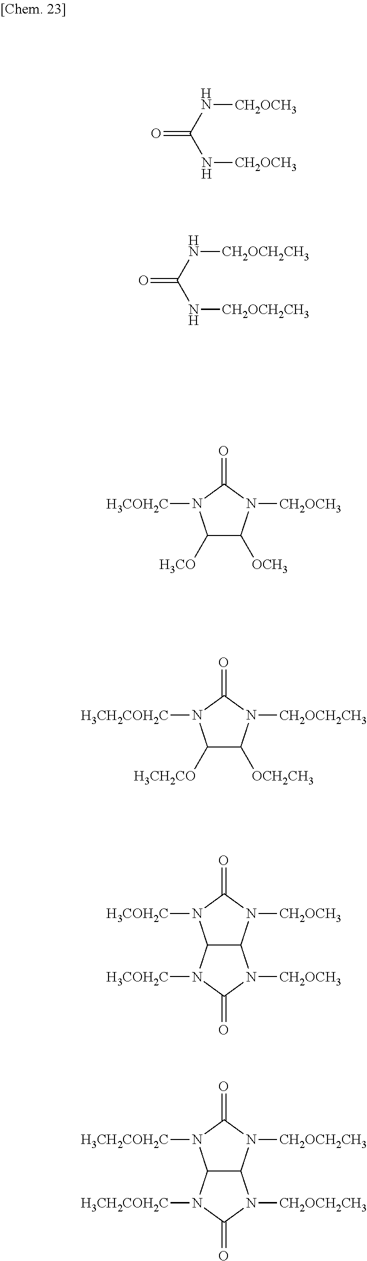

Specific examples of the compound represented by the general formula (10) include compounds illustrated below. However, the compound is not limited to these examples.

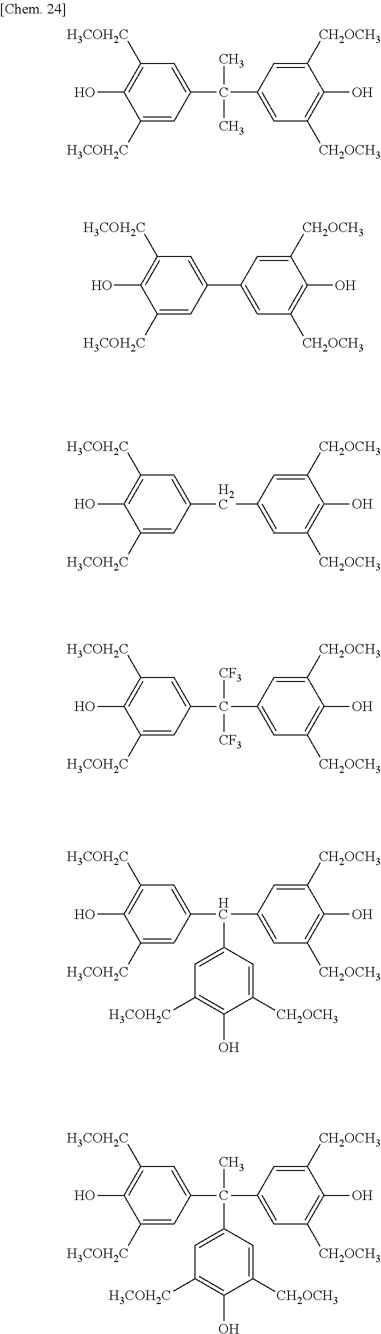

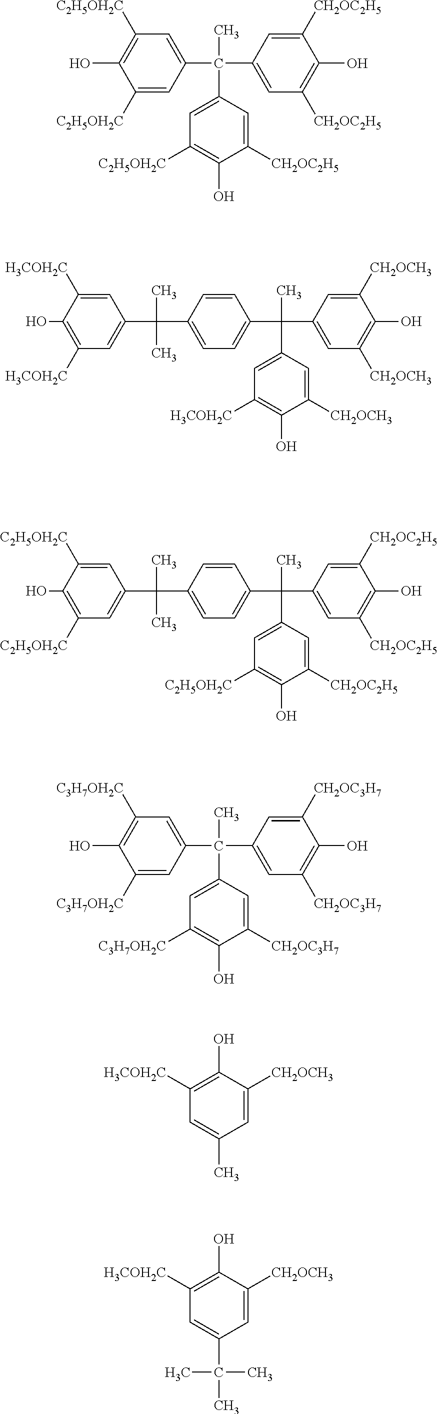

The cyclic-polyether-structure-having compound is, for example, a compound having an epoxy group or oxetanyl group. The epoxy- or oxetanyl-group-containing compound is preferably a compound having, in any molecule thereof, two or more epoxy groups or oxetanyl groups from the viewpoint of the chemical resistance and thermal resistance of the resultant cured film. Examples of the compound having, in any molecule thereof, two epoxy groups include “EPIKOTE”s (registered trade name) 807, 828, 1002, 1750 and 1007, and YX8100-BH30, E1256, E4250, and E4275 ((each showing a trade name) manufactured by Japan Epoxy Resins Co. Ltd.); “EPICLON”s (registered trade name) EXA-4880, EXA-4822, EXA-9583, and HP4032 ((each showing a trade name) manufactured by Dainippon Ink and Chemicals; “EPOLITE”s (registered trade name) 40E, 100E, 200E, 400E, 70P, 200P, 400P, 1500NP, 80MF, 4000, and 3002 ((each showing a trade name) manufactured by KYOEISHA CHEMICAL CO. LTD.; “DENACOL”s (registered trade name) EX-212L, EX-214L, EX-216L, EX-252, and EX-850L ((each showing a trade name), manufactured by Nagase ChemteX Corporation); GAN, and GOT ((each showing a trade name), manufactured by Nippon Kayaku Co., Ltd.; “CELLOXIDE” (registered trade name) 2021P ((showing a trade name), manufactured by Daicel Corporation; and “RIKARESIN”s (registered trade name) DME-100, and BEO-60E ((each showing a trade name), manufactured by New Japan Chemical Co., Ltd.). These compounds are each commercially available from the company.

Examples of the compound having three or more epoxy groups include VG3101L ((trade name), manufactured by a company Printec); “TEPIC”s (registered trade name) S, G, and P ((each showing a trade name), manufactured by Nissan Chemical Industries, Ltd.); “EPICLON”s N660, N695, and HP7200 ((each showing a trade name), manufactured by Dainippon Ink and Chemicals); “DENACOL” EX-321L ((trade name), manufactured by Nagase ChemteX Corp.); NC6000, EPPN502H, and NC3000((each showing a trade name), manufactured by Nippon Kayaku Co., Ltd.); “EPOTOTE” (registered trade name) YH-434L ((trade name), manufactured by Tohto Chemical Industry Co., Ltd.); and EHPE-3150 ((trade name), manufactured by Daicel Corporation). Examples of the compound having two or more oxetanyl groups include OXT-121, OXT-221, OX-SQ-H, OXT-191, PNOX-1009, and RSOX ((each showing a trade name), manufactured by Toa Gosei Co., Ltd.); and “ETERNACOLL”s (registered trade name) OXBP, and OXTP ((each showing a trade name), manufactured by Ube Industries, Ltd.). These compounds are each commercially available from the company.

The content of the alkoxymethyl-group-containing compound and/or cyclic-polyether-structure-having compound (e) is preferably 5 parts or more by mass for 100 parts by mass of the heat-resistant resin or heat-resistant resin precursor (a) since the resin or precursor is raised in crosslinkage density and improved in chemical resistance. When the content is 10 parts or more by mass, the resin or precursor further gains higher mechanical properties. In the meantime, the content is preferably 50 parts or less by mass, more preferably 40 parts or less by mass, even more preferably 30 parts or less by mass from the viewpoint of the storage stability and mechanical strengths of the composition.

<Colorant (f)>

The photosensitive resin composition of the present invention may further include a colorant (f).

The colorant (f) is a compound which absorbs light having specified wavelengths, and is, particularly, a compound which absorbs light having visible ray wavelengths (of 380 to 780 nm) to be colored.

The incorporation of the colorant (f) into the resin composition makes it possible to color a film obtained from the resin composition and to color, into a desired color, light rays penetrating a film of the resin composition or light rays reflected onto a film of the resin composition, or give color property thereto. Moreover, the incorporation makes it possible that light rays having wavelengths which the colorant (f) absorbs are blocked from light rays penetrating the film of the resin composition or light rays reflected onto the film of the resin composition, or that light-blocking property is given thereto.

Examples of the colorant (f) include compounds which each absorb light having visible ray wavelengths to color the resin composition into a white, red, orange, yellow, green, blue or purple. By combining two or more of these colorants with each other, light rays penetrating the film of the resin composition or light rays reflected onto the film of the resin composition can be adjusted into a desired color coordination, or can be improved in adjusted color property.

About the photosensitive resin composition of the present invention, the colorant (f) preferably includes a pigment (f1) and/or a dye (f2), which will be detailed later. The colorant (f) preferably includes a blackening agent (f3) and/or a colorant (f4) having a color other than black.

The blackening agent (f3) denotes a compound which absorbs light having visible ray wavelengths to be colored into black. The incorporation of the blackening agent (f3) into the resin composition causes a film of the resin composition to be blackened, so that this composition can be improved in light blocking property of blocking light penetrating the film of the resin composition, or blocking light reflected on the film of the resin composition. Thus, the resin composition is suitable for a film blocking film for, e.g., a black matrix of a color filter or a black column spacer of a liquid crystal display, or is suitable for any article required to be heightened in contrast by a restraint of the reflection of external light.

The blackening agent (f3) is preferably a compound which absorbs light having all visible ray wavelengths to be colored in black from the viewpoint of the light blocking property thereof. The blackening agent (f3) is also preferably a mixture of compounds in two or more colors selected from white, red, orange, yellow, green, blue and purple. By combining two or more of these colors with each other, the resin composition can be falsely colored into black to be improved in light blocking property.

About the photosensitive resin composition of the present invention, it is preferred that the blackening agent (f3) includes one or more selected from black pigments, black dyes, and mixtures each composed of dyes showing two or more colors. It is more preferred that the blackening agent (f3) includes a black pigment from the viewpoint of the light blocking property.

The colorant (f4) having a color other than black denotes a compound which absorbs light having visible ray wavelengths, so as to be colored. In other words, the colorant is a colorant colored into white, red, orange, yellow, green, blue or purple other than black.

By incorporating the blackening agent (f3) and the colorant (f4) having a color other than black into the resin composition, light blocking property, color property and adjusted color property can be given to a film of the resin composition.

About the photosensitive resin composition of the present invention, the colorant (f4) having a color other than black preferably includes a pigment having a color other than black, and/or a dye having a color other than black. The colorant (f4) more preferably includes a pigment having a color other than black from the viewpoint of the light blocking property, the thermal resistance and the weather resistance of the composition.

The content by percentage of the colorant (f) in the solid of the photosensitive resin composition of the present invention, from which the solvent is excluded, is preferably 5% or more by mass, more preferably 10% or more by mass, even more preferably 15% or more by mass. When the content by percentage is equal to or over the above-described preferred lower limit, the composition can be improved in light blocking property, color property or adjusted color property. In the meantime, the content by percentage is preferably 70% or less by mass, more preferably 65% or less by mass, even more preferably 60% or less by mass. When the content by percentage is equal to or under the above-described preferred upper limit, the composition can be improved in sensitivity when exposed to light.

<Pigment (f1)>

About the photosensitive resin composition of the present invention, the colorant (f) may include a pigment (11). An embodiment in which the colorant (f) includes the pigment (f1) is preferably an embodiment in which the composition includes the pigment (f1) as a colorant other than the blackening agent (f3) and/or the colorant (f4) having a color other than black.

The pigment (f1) denotes a compound which is physically adsorbed onto the surface of a target object, or is subjected to, e.g., interaction with the surface of a target object, thereby coloring the object. The pigment (f1) is generally insoluble in, e.g., solvents. The coloring with the pigment (f1) is high in concealing property so that the color does not easily fade by, e.g., ultraviolet rays.

By incorporating the pigment (f1) into the resin composition, the composition can be colored into a color excellent in concealing property so that a film of the resin composition can be improved in light blocking property and weather resistance.

The number-average particle diameter of the pigment (f1) is preferably from 1 to 1,000 nm, more preferably from 5 to 500 nm, even more preferably from 10 to 200 nm. When the number-average particle diameter of the pigment (f1) is in the above-described preferred ranges, improvements can be made in the light blocking property of a film of the resin composition and the dispersion stability of the pigment (f1).

The number-average particle diameter of the pigment (f1) can be obtained by measuring a laser scattering, in a solution of the pigment (f1), which is based on Brownian motion of the pigment (f1) in the solution, using a submicron particle size distribution measuring instrument (N4-PLUS, manufactured by Beckman Coulter, Inc.), or a zeta potential particle-size/molecular-weight measuring instrument (Sysmex Zetasizer Nano-ZS, manufactured by SYSMEX CORPORATION) (dynamic laser scattering method). The number-average particle diameter of the pigment (f1) in a cured film obtained from the resin composition can be obtained by measurements using an SEM and a TEM. The number-average particle diameter of the pigment (fi) is directly measured at a magnifying power of 50,000 to 200,000. When particles of the pigment (f1) are complete spheres, the respective diameter of the complete spheres are measured to gain the number-average particle diameter thereof. When particles of the pigment (f1) are not complete spheres, the following are measured: the respective longest diameters of the particles (hereinafter referred to as the “major axis diameters”); and the respective longest diameters of the particles in respective directions orthogonal to the major axis diameters (hereinafter the latter longest diameters are referred to as the “minor axis diameters”). The two-axis average value obtained by averaging the major axis diameters and the minor axis diameters is defined as the number-average particle diameter.

The pigment (f1) may be, for example, an organic pigment or inorganic pigment. The incorporation of the organic pigment into the resin composition makes it possible to give color property or adjusted color property to a film of this composition. Additionally, this pigment is an organic substance; thus, the pigment is changed in chemical structure or functionality to transmit or block light having desired specific wavelengths. By this action or some other action, the transmission spectrum or absorption spectrum of the film of the resin composition is adjusted so that the composition can be improved in adjusted color property.

Examples of the organic pigment include phthalocyanine pigments, anthraquinone pigments, quinacridone pigments, pyranthrone pigments, dioxazine pigments, thioindigo pigments, diketopyrrolopyrrole pigments, quinophthalone pigments, indanthrene pigments, indoline pigments, isoindoline pigments, isoindolinone pigments, benzofuranone pigments, perylene pigments, aniline pigments, azo pigments, azomethine pigments, condensed azo pigments, carbon black, metal complex based pigments, lake pigments, toner pigments and fluorescent pigments. Preferred are anthraquinone pigments, quinacridone pigments, pyranthrone pigments, diketopyrrolopyrrole pigments, benzofuranone pigments, perylene pigments, condensed azo pigments, and carbon black from the viewpoint of the thermal resistance thereof.

Examples of the phthalocyanine pigments include copper phthalocyanine compounds, copper halide phthalocyanine compounds, and metal-free phthalocyanine compounds.

Examples of the anthraquinone pigments includes aminoanthraquinone compounds, diaminoanthraquinone compounds, anthrapyridine compounds, flavanthrone compounds, anthanthrone compounds, indanthrone compounds, pyranthrone compounds, and violanthrone compounds.

Examples of the azo pigments include disazo compounds, and polyazo compounds.

The incorporation of the inorganic pigment into the resin composition makes it possible to give a color property or an adjusted color property to a film of the resin composition. Additionally, the pigment is an inorganic substance to be excellent in thermal resistance and weather resistance so that the film of the resin composition can be improved in thermal resistance and weather resistance.

Examples of the inorganic pigment include titanium oxide, barium carbonate, zirconium oxide, zinc flower, zinc sulfide, lead white, calcium carbonate, barium sulfate, white carbon, alumina white, silicon dioxide, kaolin clay, talc, bentonite, red oxide, molybdenum red, molybdenum orange, chrome vermilion, chrome yellow, cadmium yellow, yellow iron oxide, titanium yellow, chromium oxide, viridian, titanium cobalt green, cobalt green, cobalt chromium green, Victoria green, ultramarine blue, Prussian blue, cobalt blue, cerulean blue, cobalt silica blue, cobalt zinc silica blue, manganese violet, cobalt violet, graphite, fine particles of a metal such as silver tin alloy, titanium, copper, iron, manganese, cobalt, chromium, nickel, zinc, calcium or silver, oxides, multiple oxides, sulfides, sulfates, nitrates, carbonates, nitrides, carbides, and oxynitrides.

About the photosensitive resin composition of the present invention, the pigment (f1) is preferably a benzofuranone black pigment, and/or a perylene black pigment.

The content by percentage of the pigment (f1) in the solid of the photosensitive resin composition of the present invention, from which the solvent is excluded, is preferably 5% or more by mass, more preferably 10% or more by mass, even more preferably 15% or more by mass. When the content by percentage is equal to or over the above-described preferred lower limit, the composition can be improved in light blocking property, color property and adjusted color property. In the meantime, the content by percentage is preferably 70% or less by mass, more preferably 65% or less by mass, even more preferably 60% or less by mass. When the content by percentage is equal to or under the above-described preferred upper limit, the composition can be improved in sensitivity when exposed to light.

<Dye (f2)>

About the photosensitive resin composition of the present invention, the colorant (f) may include a dye (f2). An embodiment in which the colorant (f) includes the dye (f2) is preferably an embodiment in which the composition includes the dye (f2) as a colorant other than the blackening agent (f3) and/or the colorant (f4) having a color other than black.

The dye (f2) is a compound having therein an ionic group or a substituent such as a hydroxyl group, that is chemically adsorbed on a surface structure of a target object, or that is subjected to, e.g., a strong interaction with the same surface structure, thereby coloring the object. The dye is generally soluble in solvents, and others. In the coloring through the dye (f2), molecules thereof are individually adsorbed onto the object to give a high coloring power and a high color-developing efficiency.

The incorporation of the dye (f2) into the resin composition makes it possible to color the composition into a color excellent in coloring power, and improve a film of the resin composition in color property or adjusted color property.

Examples of the dye (f2) include direct dyes, reactive dyes, sulfide dyes, vat dyes, sulfide dyes, acid dyes, metal-containing dyes, metal-containing acid dyes, basic dyes, mordant dyes, acid mordant dyes, disperse dyes, cationic dyes, and fluorescent whiting dyes.

Examples of the dye (f2) include anthraquinone dyes, azo dyes, azine dyes, phthalocyanine dyes, methine dyes, oxazine dyes, quinoline dyes, indigo dyes, indigoid dyes, carbonium dyes, indanthrene dyes, perynone dyes, perylene dyes, triarylmethane dyes, and xanthene dyes. Preferred are the anthraquinone dyes, azo dyes, azine dyes, methine dyes, triarylmethane dyes, and xanthene dyes from the viewpoint of the solubility thereof in a solvent that will be detailed later, and the thermal resistance thereof.

The incorporation of the dye (f2) into the resin composition makes it possible to give a color property or an adjusted color property to a film of the resin composition.

The content by percentage of the dye (f2) in the solid of the photosensitive resin composition of the present invention, from which the solvent is excluded, is preferably 0.01% or more by mass, more preferably 0.05% or more by mass, even more preferably 0.1% or more by mass. When the content by percentage is equal to or over the above-described preferred lower limit, the composition can be improved in color property and adjusted color property. In the meantime, the content by percentage is preferably 50% or less by mass, more preferably 45% or less by mass, even more preferably 40% or less by mass. When the content by percentage is equal to or under the above-described preferred upper limit, the resultant cured film can be improved in thermal resistance.

### <Dispersing Agent>

The photosensitive resin composition of the present invention may further include a dispersing agent (g).

The dispersing agent (g) denotes a compound having a surface affinitive group interacting onto the surface of the pigment (f1), the disperse dye, or the like, and having a dispersion stabilizing structure for improving the dispersion stability of the pigment (f1) or the disperse dye. The dispersion stabilizing structure of the dispersing agent (g) maybe, for example, a polymer chain and/or a substituent having an electrostatic charge.

The incorporation of the dispersing agent (g) into the resin composition makes it possible that when the resin composition contains the pigment (f1) or the disperse dye, the dispersion stability thereof can be improved, and the composition can be improved in resolution after developed. When the pigment (f1) is particularly made of, for example, particles crushed into a number-average particle diameter of 1 μm or less, the particles of the pigment (f1) are increased in surface area to be easily aggregated. When the resin composition include the pigment (f1), the surfaces of the crushed pigment (f1) particles interact with the surface affinitive groups of the dispersing agent (g), and further steric hindrance and/or electrostatic repulsion is/are generated by the dispersion stabilizing structure of the dispersing agent (g), thereby making it possible to hinder the aggregation of the particles of the pigment (f1) and improve the dispersion stability of the particles.

The dispersing agent (g) that has a surface affinitive group is preferably, for example, a dispersing agent (g) having only an amine value, a dispersing agent (g) having an amine value and an acid value, a dispersing agent (g) having only an acid value, or a dispersing agent (g) having neither any amine value nor any acid value. From the viewpoint of an improvement of particles of the pigment (f1) in dispersion stability, the dispersing agent (g) is preferably the dispersing agent (g) having only an amine value, or the dispersing agent (g) having an amine value and an acid value.

About the dispersing agent (g) that has a surface affinitive group, its amine group and/or acid group serving as the surface affinitive group, preferably has/have a structure in which the group (s) is/are combined with an acid and/or a base to form one or more salts.

Examples of the dispersing agent (g) having an amine value include “DISPERBYK”s (registered trade name)-108, -109, -160, -161, -162, -163, -164, -166, -167, -168, -182, -184, -185, -2000, -2008, -2009, -2022, -2050, -2055, -2150, -2155, -2163, -2164, and -2061, and “BYK”s (registered trade name)-9075, -9077, -LP-N6919, -LP-N21116, and -LP-N21324 (each manufactured by BYK Japan K.K.); “EFKA”s (registered trade name) 4015, 4020, 4046, 4047, 4050, 4055, 4060, 4080, 4300, 4330, 4340, 4400, 4401, 4402, 4403, and 4800 (each manufactured by BASF); “AJISPER” (registered trade name) PB711 (manufactured by Ajinomoto Fine-Techno Co., Inc.); and “SOLSPERSE”s (registered trade name) 13240, 13940, 20000, 71000, and 76500 (each manufactured by the Lubrizol Corp.).

Examples of the dispersing agent (g) having an amine value and an acid value include “ANTI-TERRA”s (registered trade name)-U100 and -204, and “DISPERBYK”s (registered trade name)-106, -140, -142, -145, -180, -2001, -2013, -2020, -2025, -187 and -191, and “BYK” (registered trade name)-9076 (each manufactured by BYK Japan K.K.); “AJISPER”s (registered trade name) PB821, PB880 and PB881 (each manufactured by Ajinomoto Fine-Techno Co., Inc.); and “SOLSPERSE”s (registered trade name) 9000, 11200, 13650, 24000, 32000, 32500, 32500, 32600, 33000, 34750, 35100, 35200, 37500, 39000, 56000, and 76500 (each manufactured by the Lubrizol Corp.)

Examples of the dispersing agent (g) having only an acid value include “DISPERBYK”s (registered trade name)-102, -110, -111, -118, -170, -171, -174, -2060, and -2096, and “BYK”s (registered trade name)-P104, -P105, and -220S (each manufactured by BYK Japan K.K.); and “SOLSPERSE”s (registered trade name) 3000, 16000, 17000, 18000, 21000, 26000, 28000, 36000, 36600, 38500, 41000, 41090, 53095, and 55000 (each manufactured by the Lubrizol Corp.).

Examples of the dispersing agent (g) having neither any amine value nor any acid value include “DISPERBYK”s (registered trade name)-103, -2152, -2200 and -192 (each manufactured by BYK Japan K.K.); and “SOLSPERSE”s (registered trade name) 27000, 54000, and X300 (each manufactured by the Lubrizol Corp.).

The amine value of the dispersing agent (g) is preferably 5 mgKOH/g or more, more preferably 8 mgKOH/g or more, even more preferably 10 mgKOH/g or more. When the amine value is equal to or over the above-described preferred lower limit, the pigment (f1) can be improved in dispersion stability. In the meantime, the amine value is preferably 150 mgKOH/g or less, more preferably 120 mgKOH/g or less, even more preferably 100 mgKOH/g or less. When the amine value is equal to or under the above-described preferred upper limit, the resin composition can be improved in storage stability.

The amine value referred to herein denotes the weight of potassium hydroxide which is equivalent to the weight of an acid reacting with one gram of the dispersing agent (g). The unit thereof is mgKOH/g. This value can be obtained by neutralizing one gram of the dispersing agent (g) with an acid, and then titrating the neutralized agent with an aqueous potassium hydroxide solution. From the value of the amine value, the amine equivalent (unit: g/mol) of a resin can be obtained, which is the weight of the resin per mole of amino groups of the dispersing agent. The number of the amino groups in the dispersing agent (g) can be obtained.

The acid value of the dispersing agent (g) is preferably 5 mgKOH/g or more, more preferably 8 mgKOH/g or more, even more preferably 10 mgKOH/g or more. When the acid value is equal to or over the above-described preferred lower limit, the pigment (f1) can be improved in dispersion stability. In the meantime, the acid value is preferably 200 mgKOH/g or less, more preferably 170 mgKOH/g or less, even more preferably 150 mgKOH/g or less. When the acid value is equal to or under the above-described preferred upper limit, the resin composition can be improved in storage stability.

The acid value referred to herein is the weight of potassium hydroxide that reacts with one gram of the dispersing agent (g), and the unit thereof is mgKOH/g. The value can be obtained by titrating one gram of the dispersing agent (g) with an aqueous potassium hydroxide solution. From the acid value, the acid equivalent (unit: g/mol) of the dispersing agent (g) can be calculated out, which is the weight of the resin per mole of its acidic groups. The number of the acidic groups in the dispersing agent (g) can be obtained.

Examples of the dispersing agent (g) that has a polymer chain include acrylic resin dispersing agents, polyoxyalkylene ether dispersing agents, polyester dispersing agents, polyurethane dispersing agents, polyol dispersing agents, polyethyleneimine dispersing agents, and polyallylamine dispersing agents. From the viewpoint of a pattern processability of the resin composition by effect of alkaline developers, preferred are the acrylic resin dispersing agents, polyoxyalkylene ether dispersing agents, polyester dispersing agents, polyurethane dispersing agents, and polyol dispersing agents.

When the photosensitive resin composition of the present invention includes disperse dyes as the pigment (f1) and/or the dye (f2), the content of the dispersing agent (g) in the photosensitive resin composition of the invention is preferably 1 part or more by mass, more preferably 5 parts or more by mass, even more preferably 10 parts or more by mass in the case of regarding, as 100 parts by mass, the total amount of the pigment (f1) and/or the dye (f2), and the dispersing agent (g). When the content by percentage is equal to or over the above-described preferred lower limit, the pigment (f1) and/or the disperse dye can be improved in dispersion stability and the resin composition can be improved in resolution after developed. In the meantime, the content is preferably 60 parts or less by mass, 55 parts or less by mass, even more preferably 50 parts or less by mass. When the content is equal to or under the above-described preferred upper limit, the resultant cured film can be improved in thermal resistance.

### <Sensitizer>

The photosensitive resin composition of the present invention may further include a sensitizer.

The sensitizer denotes a compound which is exposed to light to absorb energy to generate excited triplet electrons through internal conversion and intersystem crossing, thereby aiding energy shift to, e.g., the above-mentioned photopolymerization initiator (b2).

The incorporation of the sensitizer into the resin composition makes it possible to improve the composition in sensitivity when the composition is exposed to light. It is presumed that this because the sensitizer absorbs light having long wavelengths which is not absorbed by, e.g., the photopolymerization initiator (b2), so that the energy is shifted from the sensitizer to, e.g., the photopolymerization initiator (b2), whereby the composition can be improved in photoreaction efficiency.

The sensitizer is preferably a thioxanthone sensitizer. Examples of the thioxanthone sensitizer include thioxanthone, 2-methylthioxanthone, 2-chlorothioxanthone, 2-isopropylthioxanthone, 2,4-dimethylthioxanthone, 2,4-diethylthioxanthone, and 2,4-dichlorothioxanthone.

When the total amount of the heat-resistant resin or heat-resistant resin precursor (a), and the radical polymerizable compound (d) is regarded as 100 parts by mass, the content of the sensitizer in the photosensitive resin composition of the present invention is preferably 0.01 parts or more by mass, more preferably 0.1 parts or more by mass, even more preferably 0.5 parts or more by mass, in particular preferably 1 part or more by mass. When the content is equal to or over the above-described preferred lower limit, the composition is improved in sensitivity when exposed to light. In the meantime, the content is preferably 15 parts or less by mass, more preferably 13 parts or less by mass, even more preferably 10 parts or less by mass, in particular preferably 8 parts or less by mass. When the content is equal to or under the above-described preferred upper limit, the composition can be improved in resolution after developed. Furthermore, the composition can give a slightly tapered pattern shape. <Chain Transfer Agent>

The photosensitive resin composition of the present invention may further include a chain transfer agent.

The chain transfer agent is a compound which can receive a radical from a polymer growing terminal of a polymer chain obtained by radical polymerization when the resin composition is exposed to light, thereby aiding the shift of the radical to a different polymer chain.

The incorporation of the chain transfer agent into the composition makes it possible to improve the composition in sensitivity at the light exposure. A reason therefor is presumed as follows: the radical generated by the light exposure is radical-shifted to the different polymer chain through the chain transfer agent, so that the film is radical-crosslinked down to its deep portion. For example, when the resin composition includes, particularly, the blackening agent (f3) as the colorant (f), light is absorbed into the blackening agent (f3) by the light exposure, so that the light may not reach the deep portion of the film. In the meantime, when the composition includes the chain transfer agent, the film is radical-crosslinked down to the film deep portion by radical shift through the chain transfer agent; thus, the composition can be improved in sensitivity when exposed to light.

Moreover, the incorporation of the chain transfer agent into the resin composition makes it possible to give a slightly tapered pattern shape. It is presumed that this because the radical shift through the chain transfer agent makes it possible to control the molecular weight of a polymer chain obtained by radical polymerization when the composition is exposed to light. In other words, the incorporation of the chain transfer agent hinders the production of a polymer chain having a remarkably high molecular weight, the production being on the basis of an excessive radical polymerization at the light exposure time, so as to restrain a rise in the softening point of the resultant cured film. Thus, it appears that when the composition is thermally cured, the reflowability of the resultant pattern is improved so that the composition can give a slightly tapered pattern shape.

The chain transfer agent is preferably a thiol chain transfer agent. Examples of the thiol chain transfer agent include β-mercaptopropionic acid, methyl β-mercaptopropionate, ethyl β-mercaptopropionate, 2-ethylhexyl β-mercaptopropionate, n-octyl β-mercaptopropionate, methoxybutyl β-mercaptopropionate, stearyl β-mercaptopropionte, isononyl β-mercaptopropionate, β-mercaptobutanoic acid, methyl β-mercaptobutanate, ethyl β-mercaptobutanate, 2-ethylhexyl β-mercaptobutanate, n-octyl β-mercaptobutanate, methoxybutyl β-mercaptobutanate, stearyl β-mercaptobutanate, isononyl β-mercaptobutanate, methyl thioglyconate, n-octyl thioglyconate, methoxybutyl thioglyconate, 1,4-bis(3-mercaptobutanoyloxy)butane, 1,4-bis(3-mercaptopropionyloxy)butane, 1,4-bis(thioglycoloyloxy)butane, ethylene glycol bis(thioglycolate), trimethylolethanetris(3-mercaptopropionate), trimethylolethanetris(3-mercaptobutyrate), trimethylolpropane tris(3-mercaptopropionate), trimethylolpropane tris(3-mercaptobutyrate), trimethylolpropane tris(thioglycolate), 1,3,5-tris[(3-mercaptopropionyloxy)ethyl]isocyanuric acid, 1,3,5-tris[(3-mercaptobutanoyloxy)ethyl]isocyanuric acid, pentaerythritol tetrakis(3-mercaptopropionate), pentaerythritol tetrakis(3-mercaptobutyrate), pentaerythritol tetrakis(thioglycolate), dipentaerythritol hexakis(3-mercaptopropionate), and dipentaerythritol hexakis(3-mercaptobutyrate). From the viewpoint of an improvement in the sensitivity of the resin composition when the composition is exposed to light, and a slightly tapered shape of the resultant pattern, preferred are 1,4-bis(3-mercaptobutanoyloxy)butane, 1,4-bis(3-mercaptopropionyloxy)butane, 1,4-bis(thioglycoyloxy)butane, ethylene glycol bis(thioglycolate), trimethylolethanetris(3-mercaptopropionate), trimethylolethanetris(3-mercaptobutyrate), trimethylolpropane tris(3-mercaptopropionate), trimethylolpropane tris(3-mercaptobutyrate), trimethylolpropane tris(thioglycolate), 1,3,5-tris[(3-mercaptopropionyloxy)ethyl]isocyanuric acid, 1,3,5-tris[(3-mercaptobutanoyloxy)ethyl]isocyanuric acid, pentaerythritol tetrakis(3-mercaptopropionate), pentaerythritol tetrakis(3-mercaptobutyrate), pentaerythritol tetrakis(thioglycolate), dipentaerythritol hexakis(3-mercaptopropionate), and dipentaerythritol hexakis(3-mercaptobutyrate).

When the total amount of the heat-resistant resin or heat-resistant resin precursor (a), and the radical polymerizable compound (d) is regarded as 100 parts by mass, the content of the chain transfer agent in the photosensitive resin composition of the present invention is preferably 0.01 parts or more by mass, more preferably 0.1 parts or more by mass, even more preferably 0.5 parts or more by mass, in particular preferably 1 part or more by mass. When the content is equal to or over the above-described preferred lower limit, the composition is improved in sensitivity when exposed to light, and can give a slightly tapered pattern shape. In the meantime, the content is preferably 15 parts or less by mass, more preferably 13 parts or less by mass, even more preferably 10 parts or less by mass, in particular preferably 8 parts or less by mass. When the content is equal to or under the above-described preferred upper limit, the composition can be improved in resolution after developed and can further give an improved heat-resistant cured film.

### <Polymerization Inhibitor>

The photosensitive resin composition of the present invention may further include a polymerization inhibitor.

The polymerization inhibitor is a compound which can capture a radical generated when the composition is exposed to light, or capture a radical of a polymer growing chain of a polymer chain obtained through radical polymerization at the light exposure time, thereby keeping the radical as a stable radical to stop the radical polymerization.

When an appropriate amount of the polymerization inhibitor is incorporated into the resin composition, the generation of residues is restrained after the composition is developed, and the composition is improved in resolution after the development. It is presumed that this because the polymerization inhibitor captures an excessive amount of radicals generated at the light exposure, or capture radicals of the growing terminal of the polymer chain with a high molecular weight, so that an excessive advance of the radical polymerization is restrained.

The polymerization inhibitor is preferably a phenolic polymerization inhibitor. Examples of the polymerization inhibitor include 4-methoxyphenol, 1,4-hydroquinone, 1,4-benzoquinone, 2-t-butyl-4-methoxyphenol, 3-t-butyl-4-methoxyphenol, 4-t-butylcatechol, 2,6-di-t-butyl-4-methylphenol, 2,5-di-t-butyl-1,4-hydroquinone, and 2,5-di-t-amyl-1,4-hydroquinone; and “Irganox”es (registered trade name) 1010, 1035, 1076, 098, 1135, 1330, 1726, 1425, 1520, 245, 259, 3114, 565, and 295 (each manufactured by BASF)

When the total amount of the heat-resistant resin or heat-resistant resin precursor (a), and the radical polymerizable compound (d) is regarded as 100 parts by mass, the content of the polymerization inhibitor in the photosensitive resin composition of the present invention is preferably 0.01 parts or more by mass, more preferably 0.03 parts or more by mass, even more preferably 0.05 parts or more by mass, in particular preferably 0.1 parts or more by mass. When the content is equal to or over the above-described preferred lower limit, the composition can be improved in resolution after developed and can further give an improved heat-resistant cured film. In the meantime, the content is preferably 10 parts or less by mass, more preferably 8 parts or less by mass, even more preferably 5 parts or less by mass, in particular preferably 3 parts or less by mass. When the content is equal to or under the above-described preferred upper limit, the composition can be improved in sensitivity when exposed to light.

### <Adhesion Improver>

The photosensitive resin composition of the present invention may include a silane compound to improve the adhesiveness of a product made of the composition to an underlying substrate. Specific examples of the silane compound include N-phenylaminoethyltrimethoxysilane, N-phenylaminoethyltriethoxysilane, N-phenylaminopropyltrimethoxysilane, N-phenylaminopropyltriethoxysilane, N-phenylaminobutyltrimethoxysilane, N-phenylaminobutyltriethoxysilane, vinyltrimethoxysilane, vinyltriethoxysilane, vinyltrichlorosilane, vinyltris((β-methoxyethoxy)silane, 3-methacryloxypropyltrimethoxysilane, 3-acryloxypropyltrimethoxysilane, p-styryltrimethoxysilane, 3-methacryloxypropylmethydimethoxysilane, and 3-methacryloxypropylmethyldiethoxysilane; and silane compounds described below. However, the silane compound is not limited to these examples.

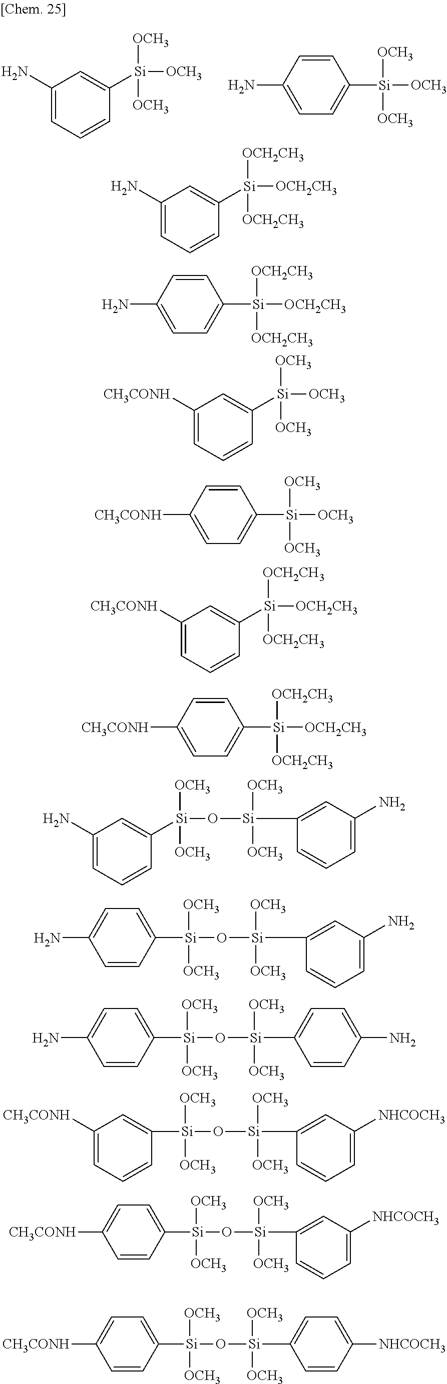

The silane compound is included in an amount that is preferably 0.001 parts or more by mass, more preferably 0.005 parts or more by mass, even more preferably 0.01 parts or more by mass for 100 parts by mass of the heat-resistant resin or heat-resistant resin precursor (a). The amount is also preferably 30 parts or less by mass, more preferably 20 parts or less by mass, even more preferably 15 parts or less by mass. When the content is in the above-described preferred ranges, the composition can produce a sufficient advantageous effect for a bonding aid while keeping thermal resistance.

### <Compound Having Phenolic Hydroxy Group>

As required, the photosensitive resin composition may include a compound having a phenolic hydroxyl group to be improved in sensitivity. When the composition includes the compound having a phenolic hydroxyl group, the resultant resin composition is hardly dissolved in any alkaline developer before exposed to light, but is easily dissolved in the alkaline developer after exposed to light. Thus, the resultant film is slightly reduced in quantity by development, and further the development is easily attained in a short period.

The content of the compound having a phenolic hydroxyl group is preferably 1 part or more by mass, more preferably 3 parts or more by mass for 100 parts by mass of the heat-resistant resin or heat-resistant resin precursor (a), and is preferably 50 parts or less by mass, more preferably 40 parts or less by mass therefor.

### <Surfactant>

As required, the photosensitive composition may include a surfactant, an ester such as ethyl lactate or propylene glycol monomethyl ether acetate, an alcohol such as ethanol, a ketone such as cyclohexanone or methyl isobutyl ketone, or an ether such as tetrahydrofuran or dioxane to be improved in wettability to a substrate. The composition may include, for example, inorganic particles of, e.g., silicon oxide or titanium oxide, or powder of polyimide.

### <Inorganic Particles>

The photosensitive resin composition used in the present invention may include inorganic particles in order, for example, to improve the dielectric constant and hardness of the resultant cured film, and decrease the thermal expansion coefficient thereof. Preferred specific examples of a material thereof include silicon oxide, titanium oxide, barium titanate, barium sulfate, barium oxide, zirconium oxide, hafnium oxide, tantalum oxide, tungsten oxide, yttrium oxide, alumina, and talc. Particularly preferred examples thereof are compounds which each have a dielectric constant (εr) of 20 or more, that is, titanium oxide (εr=115), zirconium oxide (ε=30), barium titanate (εr=400), and hafnium oxide (εr=25) to improve, particularly, the dielectric constant of the resultant cured film. However, the material of the inorganic particles is not limited to these examples. The primary particle diameter of these inorganic particles is preferably 100 nm or less, more preferably 60 nm or less.

The content of the inorganic particles is preferably from 5 to 500 parts by mass both inclusive for 100 parts by mass of the heat-resistant resin or heat-resistant resin precursor (a). When the content is in this range, the composition can express the improvement in the dielectric constant, and the other advantageous effects based on the addition of the inorganic particles while maintaining alkaline development performance.

### <Thermal Acid Generator>

The photosensitive resin composition used in the present invention may include a thermal acid generator. The thermal acid generator is heated to generate an acid, thereby promoting the crosslinking agent of a thermally crosslinking agent, and further makes it possible that when the heat-resistant resin or heat-resistant resin precursor (a) has an imide ring structure or oxazole ring structure that has not yet been ring-closed, the cyclization of the structure is promoted to improve the resultant cured film further in mechanical properties.

The thermal decomposition starting temperature of the thermal acid generator used in the present invention is preferably from 50 to 270° C., more preferably 250° C. or lower. It is preferred to select a thermal acid generator which does not generate any acid when the photosensitive resin composition of the invention is dried (or pre-baked at about 70 to 140° C.) after applied onto a substrate, but generates an acid when finally heated (or cured at about 100 to 400° C.) after exposed to light subsequently to the drying and then developed to be patterned. This is because the composition can be restrained from being lowered in sensitivity at the development time.

The acid generated from the thermal acid generator used in the present invention is preferably a strong acid. Preferred examples thereof include arylsulfonic acids such as p-toluenesulfonic acid and benzenesulfonic acid; alkylsulfonic acids such as methanesulfonic acid, ethanesulfonic acid, propanesulfonic acid and butanesulfonic acid; and haloalkylsulfonic acids such as trifluoromethylsulfonic acid. These are each used in the form of a salt such as an onium salt, or a covalent bond compound such as an imidesulfonate. The photosensitive resin composition may include two or more of these acids.

The content of the thermal acid generator used in the present invention is preferably from 0.01 to 10 parts by mass both inclusive for 100 parts by mass of the heat-resistant resin or heat-resistant resin precursor (a). When the content is in this range, the composition can express the advantageous effects based on the addition of the thermal acid generator while maintaining a high thermal resistance.

### <Production Method>

The method for producing the photosensitive resin composition of the present invention are as follows is a method of charging the components (a) to (c) and optional other components into a glass flask or stainless steel vessel, and then stirring all the components with a mechanical stirrer to dissolve soluble ones of the components; a method of dissolving the soluble components by ultrasonic waves; or a method of stirring all the components in a planetary stirring and deforming apparatus to dissolve the soluble components. The viscosity of the composition is preferably from 1 to 10000 mPa.s The resultant may be filtrated through a filter having a pore size of 0.1 to 5 μm to remove foreign materials.

The following will describe a method for forming a heat-resistant resin pattern, using the photosensitive resin composition of the present invention.

The photosensitive resin composition is applied onto a substrate. As or for the substrate, for example, the following is usable: a silicon wafer, ceramic materials, gallium arsenic, metals, glass, metal oxide insulating films, silicon nitride, and ITO. However, the substrate or the material thereof is not limited to these examples. Examples of the method for the application include spin coating, spray coating, roll coating, and slit die coating. The thickness of the composition-applied film is varied in accordance with the method for the application, the solid concentration in the composition, the viscosity of the composition, and others. Usually, the composition is applied to give a thickness of 0.1 to 150 μm after dried.

Next, the photosensitive-resin-composition-applied substrate is dried to yield a photosensitive resin film. It is preferred to perform the drying using, for example, an oven, a hot plate or infrared rays at a temperature from 50 to 150° C. for 1 minute to several hours.

Next, a chemical ray is radiated through a mask having a desired pattern onto this photosensitive resin film to expose the film to the ray. Examples of the chemical ray used for the light exposure include ultraviolet rays, visible rays, an electron beam and X-rays. In the present invention, it is preferred to use an i-line (365 nm), an h-line (405 nm) and a g-line (436 nm) from a mercury lamp.

In order to form a pattern of the heat-resistant resin from the photosensitive resin film, it is sufficient, after the light exposure, to use a developer to remove exposed portions of the film. Preferred examples of the developer include an aqueous solution of tetramethylammonium; and respective aqueous solutions of compounds each showing alkalinity. Examples of the compounds include diethanolamine, diethylaminoethanol, sodium hydroxide, potassium hydroxide, sodium carbonate, potassium carbonate, triethylamine, diethylamine, methylamine, dimethylamine, dimethylaminoethyl acetate, dimethylaminoethanol, dimethylaminoethyl methacrylate, cyclohexylamine, ethylenediamine, and hexamethylenediamine. As the case may be, to any one of these aqueous alkaline solutions may be added one selected from or any combination of two or more selected from polar solvents such as N-methyl-2-pyrrolidone, N,N-dimethylformamide, N, N-dimethylacetoamide, dimethylsulfoxide, γ-butyrolactone and dimethylacrylamide, alcohols such as methanol, ethanol and isopropanol, esters such as ethyl lactate and propylene glycol monomethyl ether acetate, and ketones such as cyclopentanone, cyclohexanone, isobutyl ketone and methyl isobutyl ketone. After the development, the composition is subjected to rinsing treatment with water. The rinsing treatment may be conducted into the state of adding, to water, for example, an alcohol such as ethanol and isopropanol, or an ester such as ethyl lactate and propylene glycol monomethyl ether acetate.

After the development, the composition is exposed to a temperature of 200 to 500° C. to be converted to a heat-resistant resin film. This heating treatment is conducted by raising the temperature of the composition up to selected temperatures step by step, or is conducted while the temperature is continuously raised in a selected temperature range for 5 minutes to 5 hours. In an example thereof, the heat treatment is conducted at 130° C., 200° C. and 350° C. for 30 minutes at each of these temperatures, or the temperature is straightly raised from room temperature to 320° C. over 2 hours.

The heat-resistant resin film formed using the photosensitive resin composition of the present invention is used suitably for a passivation film of a semiconductor, a protective film of a semiconductor element, an interlayer dielectric of a multi-layered interconnection for high-density packaging, an insulating film of an organic electroluminescent element, and others.

### EXAMPLES

Hereinafter, the present invention will be described in detail by way of working examples thereof. However, the invention is not limited by these examples. In each of the working examples, the production and evaluation of a cured film were performed by methods described below.

### <Pattern Processability Evaluation>

(1) Production of Photosensitive Resin Film

A photosensitive resin composition (hereinafter referred to as a varnish) was applied onto a 6-inch silicon wafer. Next, a hot plate (application and development apparatus Mark-7, manufactured by Tokyo Electron Limited) was used to prebake the resultant workpiece at 120° C. for 3 minutes to yield a photosensitive resin film.
(2) Method for Measuring Film Thickness

A product, Lambda ACE STM-602, manufactured by Dainippon Screen Mfg. Co., Ltd. was used to measure the thickness of the pre-baked film, and that of a film obtained by developing the pre-baked film at a refractive index of 1.629, and measure that of a cured film obtained by curing the developed film at a refractive index of 1.773.
(3) Exposure to Light

A reticle cut into a pattern was set to an exposure apparatus (i-line stepper DSW-8570i, manufactured by GCA Corp.), and the photosensitive resin film was exposed to an i-line at an intensity of 365 nm while the period for the exposure was changed.
(4) Development

A development apparatus, Mark-7, manufactured by Tokyo Electron Limited was used to develop the light-exposed film two times with a 2.38% solution of tetramethylammonium hydroxide in water for 45 seconds. Next, the workpiece was subjected to rinsing treatment with pure water. The workpiece was shaken to remove the water, and dried.
(5) Sensitivity Calculation

The following was defined as the sensitivity of the film: the exposure value at which the light-exposed portions were dissolved to be completed lost (the value is referred to as the minimum exposure value Eth) after the exposure and the development.
(6) Resolution Calculation

After the exposure and the development, the following was defined as the resolution of the film: at the Eth, a pattern width permitting a line-and-space pattern (1L/1S) to be formed with a one-to-one width.
(7) Cured Film Production

An inert gas oven INH-21CD manufactured by Koyo Thermo Systems Co., Ltd. was used to heat the resin film produced by the above-mentioned method at 140° C. in a nitrogen gas flow (oxygen concentration: 20 ppm) for 30 minutes, and then the temperature of the film was raised to 230° C. in a period of 30 minutes. At 230° C., the film was thermally treated for 1 hour to produce a cured film (heat-resistant resin film).

### <Chemical Resistance Evaluation>

The cured film produced by the above-mentioned method was subjected to immersing treatment in a peeling liquid 106 manufactured by Tokyo Ohka Kogyo Co., Ltd. at 70° C. for 10 minutes, About the treated cured film, the respective film thicknesses of the film before and after the treatment were measured and then the amount of the reduced film was gained.

### <Thermal Resistance Evaluation>

The cured film produced by the above-mentioned method on the silicon wafer was peeled with hydrofluoric acid to yield a film. Into an aluminum clamp cell was filled 10 mg of this simple film, so as to produce a TGA measuring sample, and an apparatus TGA-50 (manufactured by SHIMADZU CORPORATION) was used to make a measurement. In a nitrogen atmosphere, the temperature at which the weight of the sample was reduced in a proportion of 5% was measured from 200° C. When the sample was a sample about which the temperature was lower than 320° C., the sample was judged not to be satisfactory (cross mark). Alternatively, when the sample was a sample about which the temperature was 320° C. or higher, the sample was judged to be good (circular mark).

### Synthesis Example 1

### Synthesis of Bisaminophenol Compound (a)

Into 250 mL of chloroform was dissolved 10.0 g (0.0598 mol) of 2,6-dihydroxymethyl-4-methylphenol. Thereto was added 36.0 g (0.414 mol) of manganese dioxide to cause the reactive components to react with each other at 60° C. for 20 hours. The reaction solution was filtrated, and the filtrate was dried under a reduced pressure. Thereafter, the precipitated yellow solid was caused to undergo reaction at 230° C. for 1 hour in the presence of 55.0 g (0.98 mol) of potassium hydroxide. The system was cooled to room temperature and the mixture was dissolved into 150 mL of pure water. The resultant was filtrated, and hydrochloric acid was added to the filtrate until the pH of the filtrate turned to 1. The precipitation was filtrated. The filtrate was washed with pure water, and dried at 110° C. all night to yield a yellowish brown solid. This solid was stirred at room temperature in 110 mL of thionyl chloride for 2 hours, and the resultant was subjected to filtration. The filtrate was dried under a reduced pressure to yield a brown solid.

38.5 g (0.105 mol of bis (3-amino-4-hydroxyphenyl) hexafluoropropane (BAHF) was dissolved into 200 mL of acetone and 17.4 g (0.3 mol) of propylene oxide. Thereto was dropwise added a solution in which 11.7 g (0.05 mol) of the brown solid yielded previously was dissolved in 100 mL of acetone. After the addition, the reactive components were caused to react with each other at room temperature for 4 hours, and then the precipitated white solid was collected by filtration. The solid was vacuum-dried at 50° C. to yield a bisaminophenol compound (a) represented by the following formula:

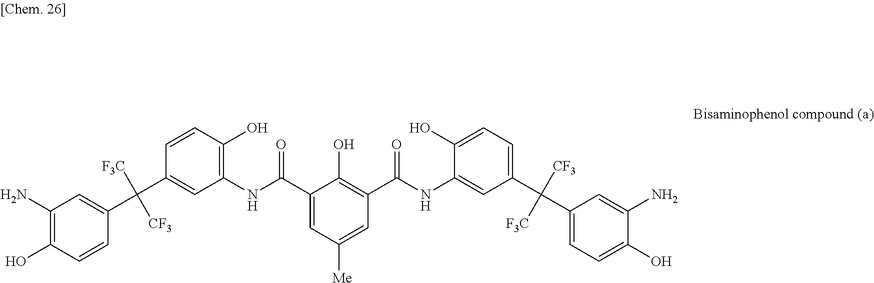 [SMILES: Cc1cc(C(=O)Nc2cc(C(c3ccc(O)c(N)c3)(C(F)(F)F)C(F)(F)F)ccc2O)c(O)c(C(=O)Nc2cc(C(c3ccc(O)c(N)c3)(C(F)(F)F)C(F)(F)F)ccc2O)c1]

### Synthesis Example 2

### Synthesis of Bisaminophenol Compound (b)

The same manner as in Synthesis Example 1 was carried out except the use of 17.2 g (0.0598 mol) of bis(2-hydroxy-3-hydroxymethyl-5-methylphenol)methane instead of 10.0 g (0.0598 mol) of 2,6-dihydroxymethyl-4-methylphenol. In this way, a bisaminophenol compound (b) was yielded.

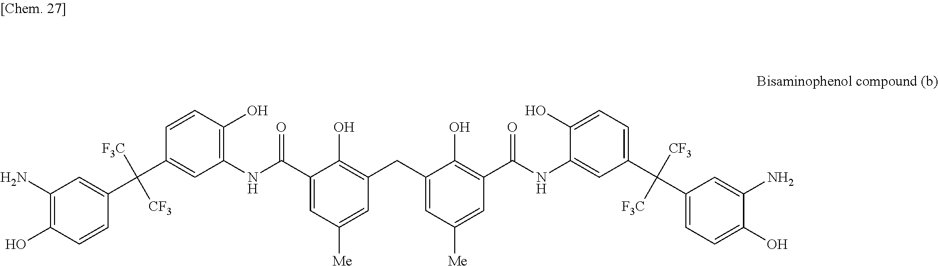 [SMILES: Cc1cc(Cc2cc(C)cc(C(=O)Nc3cc(C(c4ccc(O)c(N)c4)(C(F)(F)F)C(F)(F)F)ccc3O)c2O)c(O)c(C(=O)Nc2cc(C(c3ccc(O)c(N)c3)(C(F)(F)F)C(F)(F)F)ccc2O)c1]

### Synthesis Example 3

### Synthesis of Polyimide Precursor (Polymer A)

In a dry nitrogen gas flow, 21.4 g (0.024 mol) of the bisaminophenol compound (a) yielded in Synthesis Example 1 and 0.37 g (0.002 mol) of 1,3-bis(3-aminopropyl)tetramethyldisiloxane (SiDA) were dissolved into 80 g of N-methyl-2-pyrrolidone (NMP). Thereto was added 9.31 g (0.030 mol) of 3,3′, 4,4′-diphenyl ether tetracarboxylic acid anhydride (ODPA) together with 10 g of NMP to cause the reactive components to react with each other at 40° C. for 1 hour. Thereafter, thereto was added 0.65 g (0.006 mol) of 3-aminophenol as a terminal blocking agent, and further the reactive components were caused to react with each other at 40° C. for 1 hour. Thereafter, over 10 minutes, thereto was dropwise added a solution in which 7.14 g (0.06 mol) of N,N′-dimethylformamide dimethylacetal was diluted with 15 g of NMP. After the addition, the reaction system was stirred at 40° C. for 2 hours. After the end of the reaction, the solution was charged into 2 L of water, and the resultant polymer solid precipitation was collected by filtration. The polymer solid was dried in a vacuum drier of 50° C. for 72 hours to yield a polymer A as a polyimide precursor.

### Synthesis Example 4

### Synthesis of Polyimide Precursor (Polymer B)

The same manner as in Synthesis Example 1 was carried out except the use of 24.4 g (0.024 mol) of the bisaminophenol compound (b) yielded in Synthesis Example 2 instead of 21.4 g (0.024 mol) of the bisaminophenol compound (a) yielded in Synthesis Example 1. In this way, a polymer (B) as a polyimide precursor was yielded.

### Synthesis Example 5

### Synthesis of Polyimide (Polymer C)

In a dry nitrogen gas flow, 80.3 g (0.09 mol) of the bisaminophenol compound (a) yielded in Synthesis Example 1 was dissolved into 500 g of NMP. Thereto was added 31.0 g (0.1 mol) of ODPA together with 50 g of NMP, and the reaction solution was stirred at 30° C. for 2 hours. Thereafter, thereto was added 2.18 g (0.02 mol) of 3-aminophenol, and further the solution was continuously stirred at 40° C. for 2 hours. Furthermore, 5 g of pyridine was diluted with 30 g of toluene, and this liquid was added to the solution. A condenser was attached to the reaction system. While water was removed together with toluene to the outside of the system by azeotropy, the reactive components were caused to react with each other for 2 hours in the state of setting the temperature of the solution to 120° C., and further the reaction was conducted for 2 hours at 180° C. When the temperature of this solution was lowered to room temperature, the solution was charged into 3 L of water to yield a white powder. This powder was collected by filtration, and further washed 3 times with water. After the washing, the white powder was dried in a vacuum drier of 50° C. for 72 hours to yield a polyimide polymer C.

### Synthesis Example 6

### Synthesis of Polyhydroxyamide (Polymer D)

In a dry nitrogen gas flow, 26.8 g (0.03 mol) of the bisaminophenol compound (a) and 7.3 g (0.02 mol) of BAHF were dissolved into 50 g of NMP and 26.4 g (0.3 mol) of glycidyl methyl ether. The temperature of the solution was cooled to −15° C. Thereto was dropwise added a solution in which 14.7 g (0.050 mol) of diphenyl ether dicarboxylic acid dichloride was dissolved in 25 g of GBL in such a manner that the internal temperature did not exceed 0° C. After the end of the addition, the stirring of the solution was continued at −15° C. for 6 hours. After the end of the reaction, the solution was charged into 3 L of water containing 10% by mass of methanol, and the resultant white precipitation was collected. This precipitation was collected by filtration, and washed 3 times with water. Thereafter, the white powder was dried in a vacuum drier of 50° C. for 72 hours to yield a polyhydroxyamide polymer D.

### Comparative Synthesis Example 1

### Synthesis of Polyimide Precursor (Polymer E)

In a dry nitrogen gas flow, 8.8 g (0.024 mol) of BAHF and 0.37 g (0.002 mol) of SiDA were dissolved into 80 g of NMP. Thereto was dropwise added 9.31 g (0.030 mol) of ODPA together with 10 g of NMP, and the reactive components were caused to react with each other at 40° C. for 1 hour. Thereafter, thereto was added 0.65 g (0.006 mol) of 3-aminophenol as a terminal blocking agent, and further reaction was conducted at 40° C. for 1hour. Thereafter, over 10minutes, thereto was dropwise added a solution in which 7.14 g (0.06 mol) of N,N-dimethylformamide dimethylacetal was diluted with 15 g of NMP. After the addition, the solution was stirred at 40° C. for 2 hours. After the end of the reaction, the solution was charged into 2 L of water, and the resultant polymer solid precipitation was collected by filtration. The polymer solid was dried in a vacuum drier of 50° C. for 72 hours to yield a polyimide precursor polymer E.

### Comparative Synthesis Example 2

### Synthesis of Polyimide (Polymer F)

In a dry nitrogen gas flow, 32.9 g (0.09 mol) of BAHF was dissolved into 500 g of NMP. Thereto was added 31.0 g (0.1 mol) of ODPA together with 50 g of NMP, and the reaction solution was stirred at 30° C. for 2 hours. Thereafter, thereto was added 2.18 g (0.02 mol) of 3-aminophenol, and further the solution was continuously stirred at 40° C. for 2 hours. Furthermore, 5 g of pyridine was diluted with 30 g of toluene (manufactured by Tokyo Chemical Industry Co., Ltd.), and this liquid was added to the solution. A condenser was attached to the reaction system. While water was removed together with toluene to the outside of the system by azeotropy, the reactive components were caused to react with each other for 2 hours in the state of setting the temperature of the solution to 120° C., and further the reaction was conducted for 2 hours at 180° C. When the temperature of this solution was lowered to room temperature, the solution was charged into 3 L of water to yield a white powder. This powder was collected by filtration, and further washed 3 times with water. Thereafter, the white powder was dried in a vacuum drier of 50° C. for 72 hours to yield a polyimide polymer F.

### Synthesis Example 7

### Synthesis of Quinonediazide Compound (c)

Ina dry nitrogen gas flow, 21.22 g (0.05 mol) of a product Tris P-PA ((trade name) manufactured by Honshu Chemical Industry Co., Ltd.), 26.86 g (0.10 mol) of 5-naphthoquinonediazidesulfonic acid chloride, and 13.43 g (0.05 mol) of 4-naphthoquinonediazidesulfonic acid chloride were dissolved into 450 g of 1,4-dioxane. The temperature of the solution was adjusted to room temperature. In this solution, 15.18 g of triethylamine was used which was mixed with 50 g of 1,4-dioxane. The same manner as in Synthesis Example 6 was carried out to yield a quinonediazide compound (c) represented by the following formula:

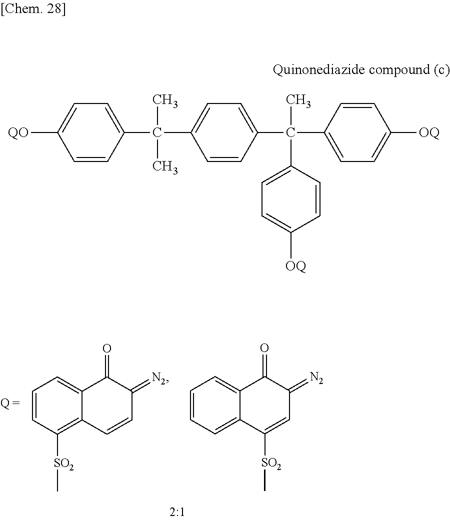

### Synthesis Example 8

### Synthesis ofAlkoxymethyl-Group-Containing Thermally Crosslinking Agent (d)

(1) 103.2 g (0.4 mol) of the product Tris P-HAP was dissolved into the solution of 80 g (2.0 mol) of sodium hydroxide in 800 g of pure water. After the product was completely dissolved, over 2 hours thereto was dropwise added 686 g of an aqueous formalin solution having a concentration of 36 to 38% by mass at 20 to 25° C. Thereafter, the solution was stirred at 20 to 25° C. for 17 hours. Thereto were added 98 g of sulfuric acid and 552 g of water to neutralize the solution. The solution was allowed to stand still as it was for 2 days. After the standing-still, a needle-form white crystal generated in the solution was collected by filtration, and washed with 100 mL of water. This white crystal was vacuum-dried at 50° C. for 48 hours. The dried white crystal was analyzed at 254 nm by a high performance liquid chromatography manufactured by SHIMADZU CORPORATION, using an ODS as a column, and acetonitrile and water (=70/30) as an eluent. As a result, it was understood that the starting materials were completely lost and the purity of the crystal was 92%. Furthermore, DMSO-d6 was used as a deuterated solvent to analyze the crystal by NMR (GX-270, manufactured by JEOL Ltd.). As a result, it was understood that the crystal was a hexa-methyloled Tris P-HAP.
(2) Next, the thus yielded compound was dissolved into 300 mL of methanol. Thereto was added 2 g of sulfuric acid, and the solution was stirred at room temperature for 24 hours. To this solution was added 15 g of an anionic ion exchange resin (AMBERLYST IRA96SB, manufactured by the company Rohm and Haas), and the resultant was stirred for 1 hour. The ion exchange resin was removed by filtration. Thereafter, thereto was added 500 mL of ethyl lactate. A rotary evaporator was used to remove methanol to convert the solution to an ethyl lactate solution. This solution was allowed to stand still at room temperature for 2 days. As a result, a white crystal was generated. The resultant white crystal was analyzed by high performance liquid chromatography. As a result, it was understood that the compound of the crystal was a 99%-purity hexamethoxymethyl compound (alkoxymethyl-group-containing thermally crosslinking agent (d)) of the Tris P-HAP. This is represented by the following formula:

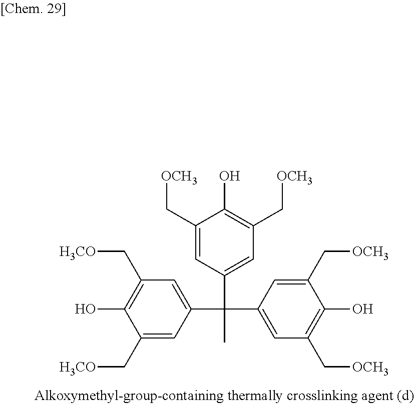 [SMILES: COCc1cc(C(C)(c2cc(COC)c(O)c(COC)c2)c2cc(COC)c(O)c(COC)c2)cc(COC)c1O]

Different thermally crosslinking agents and a different compound having a phenolic hydroxide group that were each used in the working examples are as follows:

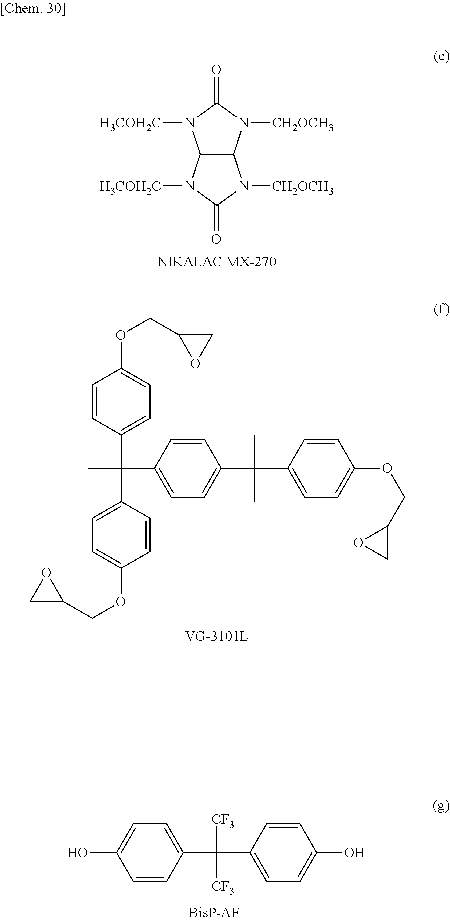

### Example 1

The following were each weighed out: 10 g of the polymer A solid yielded in Synthesis Example 3; 1.9 g of the quinonediazide compound (c) yielded in Synthesis Example 8; 1.2 g of the alkoxymethyl-group-containing thermally crosslinking agent (d) yielded in Synthesis Example 9; and 0.6 g of the phenolic compound (g). These components were dissolved into 30 g of GBL to yield a varnish of a positive photosensitive resin composition. The resultant varnish was used to make the individual evaluations by the above-mentioned respective methods.

### Example 2

A vanish of a positive photosensitive resin composition was yielded in the same way as in Example 1 except the use of 10 g of the polymer B solid yielded in Synthesis Example 4 instead of the polymer A, and 1.2 g of the NIKALAC MX-270 (e) instead of the alkoxymethyl-group-containing thermally crosslinking agent (d). The evaluations of the varnish were made by the respective methods.

### Example 3

A vanish of a positive photosensitive resin composition was yielded in the same way as in Example 1 except the use of 10 g of the polymer C solid yielded in Synthesis Example 5 instead of the polymer A, and 1.2 g of the VG-3101L (f) instead of the alkoxymethyl-group-containing thermally crosslinking agent (d). The evaluations of the varnish were made by the respective methods.

### Example 4

A vanish of a positive photosensitive resin composition was yielded in the same way as in Example 1 except the use of 10 g of the polymer D solid yielded in Synthesis Example 6 instead of the polymer A. The evaluations of the varnish were made by the respective methods.

### Example 5

A vanish of a positive photosensitive resin composition was yielded in the same way as in Example 1 except the use of 1.2 g of the VG-3101L (f) instead of the alkoxymethyl-group-containing thermally crosslinking agent (d). The evaluations of the varnish were made by the respective methods.

### Example 6

The following were each weighed out: 10 g of the polymer A solid yielded in Synthesis Example 3; 1.4 g of 1-(9-ethyl-6-nitro-9H-carbazole-3-yl)-1-[2-methyl-4-(1-meth oxypropane-2-yloxy)phenyl]methanone-1-(O-acetyl)oxime) (NCI-831 “ADEKAARKLS” (registered trade name)) (manufactured by Adeka Corp.); 4.0 g of dipentaerythritol hexaacrylate (DPHA) (manufactured by Nippon Kayaku Co., Ltd.); and 1.2 g of the alkoxymethyl-group-containing thermally crosslinking agent (d) yielded in Synthesis Example 9. These components were dissolved into 30 g of GEL to yield a varnish of a negative photosensitive resin composition. The resultant varnish was used to make the individual evaluations by the above-mentioned respective methods.

### Comparative Example 1

A vanish of a positive photosensitive resin composition was yielded in the same way as in Example 1 except the use of 10 g of the polymer E solid yielded in Comparative Synthesis Example 1 instead of the polymer A. The evaluations of the varnish were made by the respective methods.

### Comparative Example 2

A vanish of a positive photosensitive resin composition was yielded in the same way as in Example 3 except the use of 10 g of the polymer F solid yielded in Comparative Synthesis Example 2 instead of the polymer C. The evaluations of the varnish were made by the respective methods.

### Comparative Example 3

The following were each weighed out: 8 g of the polymer E solid yielded in Comparative Synthesis Example 1; 2 g of a Novolak resin G (trade name: PSF-2808, manufactured by Gunei Chemical Industry Co., Ltd.; m/p ratio: 100/0); 1.9 g of the quinonediazide compound (c) yielded in Synthesis Example 8; 1.2 g of the alkoxymethyl-group-containing thermally crosslinking agent (d) yielded in Synthesis Example 9; and 0.6 g of the phenolic compound (g). These components were dissolved into 30 g of GEL to yield a varnish of a positive photosensitive resin composition. The resultant varnish was used to make the individual evaluations by the above-mentioned respective methods.

### Comparative Example 4

A vanish of a positive photosensitive resin composition was yielded in the same way as in Comparative Example 3 except the use of 2 g of a Novolak resin H (trade name: XPS-4958D, manufactured by Gunei Chemical Industry Co., Ltd.; m/p ratio: 45/55) instead of the Novolak resin G, and 1.2 g of the NIKALAC MX-270 (e) instead of the alkoxymethyl-group-containing thermally crosslinking agent (d). The evaluations of the varnish were made by the respective methods.

About each of Examples 1 to 5, and Comparative Examples 1 to 4, the composition thereof is shown in Table 1, and the individual evaluation results are shown in Table 2.

TABLE 1

| | | | | Alkoxymethyl coumpond | | |
|---|---|---|---|---|---|---|
| | | | | (e) or coumpomd (e) | | |
| | | Photosensitive | | having a cyclic | | Different |
| | Polymer (a) | compound (b) | Solvent (c) | polyether structure | Novolak resin | component |
| Example 1 | A: 10 g | c: 1.9 g | GBL: 30 g | d: 1.2 g | — | g: 0.6 g |
| Example 2 | B: 10 g | c: 1.9 g | GBL: 30 g | e: 1.2 g | — | g: 0.6 g |
| Example 3 | C: 10 g | c: 1.9 g | GBL: 30 g | f: 1.2 g | — | g: 0.6 g |
| Example 4 | D: 10 g | c: 1.9 g | GBL: 30 g | d: 1.2 g | — | g: 0.6 g |
| Example 5 | A: 10 g | c: 1.9 g | GBL: 30 g | f: 1.2 g | — | g: 0.6 g |
| Example 6 | A: 10 g | NCI-831: 1.4 g | GBL: 30 g | d: 1.2 g | — | DPHA: 4.0 g |
| Comparative Example 1 | E: 10 g | c: 1.9 g | GBL: 30 g | d: 1.2 g | — | g: 0.6 g |
| Comparative Example 2 | F: 10 g | c: 1.9 g | GBL: 30 g | f: 1.2 g | — | g: 0.6 g |
| Comparative Example 3 | E: 8 g | c: 1.9 g | GBL: 30 g | d: 1.2 g | Novolak G: 2 g | g: 0.6 g |
| Comparative Example 4 | E: 8 g | c: 1.9 g | GBL: 30 g | e: 1.2 g | Novolak H: 2 g | g: 0.6 g |

TABLE 2

| | Pattern processibilities | | Chemical resistance | |
|---|---|---|---|---|
| | Sensitivity | Resolution | Reduced film | Heat |
| | [mJ/cm²] | [μm] | quantity [μm] | resistance |
| Example 1 | 160 | 3 | 0.15 | ∘ |
| Example 2 | 150 | 2 | 0.20 | ∘ |
| Example 3 | 200 | 4 | 0.12 | ∘ |
| Example 4 | 150 | 2 | 0.20 | ∘ |
| Example 5 | 150 | 2 | 0.20 | ∘ |
| Example 6 | 190 | 6 | 0.18 | ∘ |
| Comparative | 250 | 5 | 0.20 | ∘ |
| Example 1 | | | | |
| Comparative | 350 | 6 | 0.18 | ∘ |
| Example 2 | | | | |
| Comparative | 160 | 3 | 1.20 | x |
| Example 3 | | | | |
| Comparative | 180 | 4 | 1.00 | x |
| Example 4 | | | | |

1-13. (canceled)

14. A heat-resistant resin or heat-resistant resin precursor, having a structure originating from the diamine represented by the general fonnula (1): 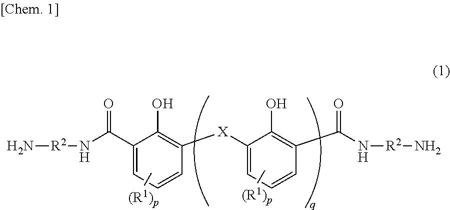
wherein each R¹ represents an alkyl group having 1 to 5 carbon atoms; each p represents an integer of 0 to 2; q represents an integer of 0 to 100; each R² represents any case of a bivalent aliphatic group, alicyclic group or aromatic group, any case of a bivalent organic group in which plural aromatic groups are bonded to each other through a single bond, or any case of a bivalent organic group in which plural aromatic groups are bonded to each other through —O—, —CO—, —SO₂—, —CH₂—, —C(CH₃)₂—, or —C(CF₃)₂ wherein each F is each fluorine; X represents —O—, —S—, —CO—, —SO₂—, —CH₂—, —C(CH₃)₂—, —C(CH₃)(C₂H₅)—, or —C(CF₃)₂— wherein each F is fluorine.

15. The heat-resistant resin or heat-resistant resin precursor according to claim 14, including at least one selected from polyimides, polybenzoxazoles, polybenzoimidazoles, and polybenzothiazoles; and respective precursors of these polymers, and copolymers of these polymers.

16. The heat-resistant resin or heat-resistant resin precursor according to claim 14, having at least one selected from respective structures represented by the general formulae (2), (3), and (5): 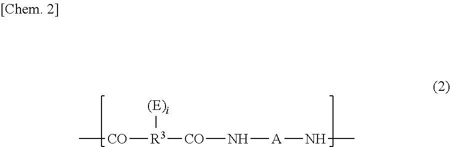
wherein R³ represents a bivalent to hexavalent organic group having 2 to 30 carbon atoms; E represents any one of OR⁴, SO₃R⁴, CONR⁴R⁵, COOR⁴, and SO₂NR⁴R⁵ wherein R⁴ and R⁵ each independently represent a hydrogen atom or a monovalent hydrocarbon group having 1 to 20 carbon atoms; i represents an integer of 0 to 4; and A represents a structure represented by a general formula (4); 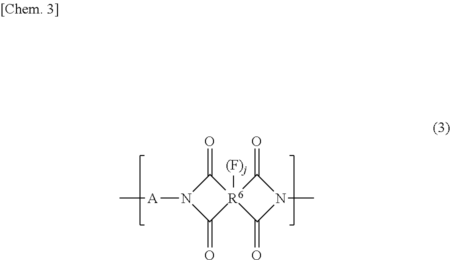
wherein R⁶ represents a tetravalent to octavalent organic group having 2 to 30 carbon atoms; F represents any one of OR⁷, SO₃R⁷, CONR⁷R⁸, COOR⁷, and SO₂NR⁷R⁸ wherein R⁷ and R⁸ each independently represent a hydrogen atom or a monovalent hydrocarbon group having 1 to 10 carbon atoms; j represents an integer of 0 to 4; and A represents a structure represented by the general formula (4): 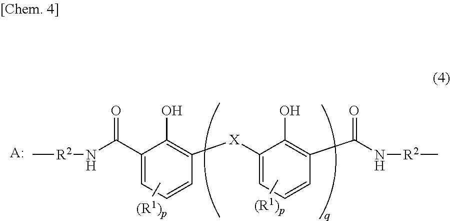
wherein each R¹ represents an alkyl group having 1 to 5 carbon atoms; each p is an integer of 0 to 2; q represents an integer of 0 to 100; each R² represents any case of a bivalent aliphatic group, alicyclic group, or aromatic group, any case of a bivalent organic group in which plural aromatic group are bonded to each other through a single bond, or any case of a bivalent organic group in which plural aromatic groups are bonded to each other through —O—, —CO—, —SO₂—, —CH₂—, —C(CH₃)₂—, or —C(CF₃)₂ wherein each F is fluorine; and X represents —O—, —S—, —CO—, —SO₂—, —CH₂—, —C(CH₃)₂—, —C(CH₃)(C₂H₅)—, or —C(CF₃)₂ wherein each F is fluorine; and 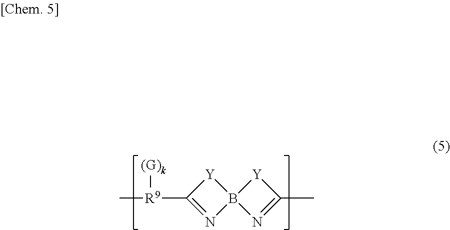
wherein R⁹ represents a bivalent to hexavalent organic group having 2 to 30 carbon atoms; G represents any one of OR¹⁰, SO₃R¹⁰, CONR¹⁰R¹¹, COOR¹⁰, and SO₂NR¹⁰R¹¹ wherein R¹⁰ and R¹¹ each represent a hydrogen atom or a monovalent hydrocarbon group having 1 to 10 carbon atoms; k represent an integer of 0 to 4; B represents a structure represented by a general formula (6), and each Y represents NH, O or S; 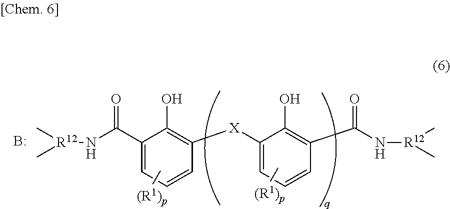
wherein each R¹ represents an alkyl group having 1 to 5 carbon atoms; each p represents an integer of 0 to 2; q represents an integer of 0 to 100; each R¹² represents any case of a trivalent aliphatic group, alicyclic group, or aromatic group, any case of a trivalent organic group in which plural aromatic groups are bonded to each other through a single bond, or any case of a trivalent organic group in which plural aromatic groups are bonded to each other through —O—, —CO—, —SO₂—, —CH₂—, —C(CH₃)₂—, or —C(CF₃)₂ wherein each F is fluorine; and X represents —O—, —S—, —CO—, —SO₂—, —CH₂—, —C(CH₃)₂—, —C(CH₃)(C₂H₅)—, or —C(CF₃)₂.

17. A photosensitive resin composition, comprising the heat-resistant resin or heat-resistant resin precursor (a) recited in claim 14, and further comprising a photosensitive compound (b) and a solvent (c).

18. The photosensitive resin composition according to claim 17, wherein the photosensitive compound (b) is a quinonediazide compound (b1).

19. The photosensitive resin composition according to claim 17, wherein the photosensitive compound (b) is a photopolymerization initiator (b2).

20. The photosensitive resin composition according to claim 19, further comprising a radical polymerizable compound (d).

21. The photosensitive resin composition according to claim 17, further comprising an alkoxymethyl-group-containing compound, and/or a cyclic-polyether-structure-having compound (e).

22. A cured film, wherein the photosensitive resin composition recited in claim 17 is cured.

23. An element, comprising the cured film recited in claim 22.

24. An organic EL display device, wherein the cured film recited in claim 22 is located over at least one of a planarizing layer over a driving circuit, and an insulation layer over a first electrode.

25. A method for producing an organic EL display device, using the photosensitive resin composition recited in claim 17, comprising:
the step of applying the photosensitive resin composition onto a substrate to form a photosensitive resin film; and
the step of subjecting the photosensitive resin film to drying, exposure to light, development, and heating treatment.
